(19) United States
(12) Patent Application Publication
(10) Pub. No.: US 2022/0002227 A1
(43) Pub. Date: Jan. 6, 2022

(54) CRYSTALLINE FORMS OF METYROSINE

(71) Applicant: Tyme, Inc., Bedminster, NJ (US)

(72) Inventor: John Zucaro, Newark, DE (US)

(73) Assignee: Tyme, Inc., Bedminster, NJ (US)

(21) Appl. No.: 17/365,356

(22) Filed: Jul. 1, 2021

Related U.S. Application Data
(60) Provisional application No. 63/046,893, filed on Jul. 1, 2020.

(51) Int. Cl. C07C 229/36; C07C 227/42

(52) U.S. Cl. CPC C07C 229/36; C07B 2200/13; C07C 227/42

(57) ABSTRACT

The disclosure provides crystalline compositions comprising D-metyrosine and L-metyrosine, pharmaceutical formulation comprising one or more crystalline composition described herein and a pharmaceutically acceptable excipient, methods for treating cancer in a patient in need thereof, the method comprising administering to the patient one or more composition described herein, and methods for preparing the compositions described herein.

### CROSS-REFERENCE TO RELATED APPLICATIONS

This application claims the benefit of the priority of U.S. Provisional Patent Application No. 63/046,893, filed Jul. 1, 2020, the contents of which are herein incorporated by reference.

### TECHNICAL FIELD

This invention relates to crystalline compositions comprising D-, L-metyrosine, or D,L-metyrosine, methods of using the crystalline compositions, and methods of preparing the crystalline compositions.

### BACKGROUND

Metyrosine is an inhibitor of the enzyme tyrosine hydroxylase and depletes levels of the catecholamines, such as dopamine, adrenaline and noradrenaline, when administered to patients. L-metyrosine is useful in the treatment high blood pressure in patients having pheochromocytoma, an adrenal gland cancer.

What is needed are alternate compositions comprising metyrosine.

### SUMMARY

In some aspects, the disclosure provides crystalline compositions comprising about 55% to about 95% by weight of D-metyrosine and about 5% to about 45% by weight of L-metyrosine, based on the total weight of the crystalline composition, and is characterized by a powder X-ray diffraction pattern comprising at least three peaks selected from about 10.2 degrees 2 θ, about 13.5 degrees 2 θ, about 20.1 degrees 2 θ, about 21.5 degrees 2 θ, about 22.5 degrees 2 θ, or about 28.0 degrees 2 θ.

In other aspects, the disclosure provides crystalline compositions comprising about 55% to about 95% by weight of L-metyrosine and about 5% to about 45% by weight of D-metyrosine, based on the total weight of the crystalline composition, and characterized a powder X-ray diffraction pattern comprising at least three peaks selected from about 9.9 degrees 2 θ, about 13.4 degrees 2 θ, about 16.4 degrees 2 θ, about 20.0 degrees 2 θ, or about 21.9 degrees 2 θ.

In further aspects, the disclosure provides pharmaceutical formulations comprising one or more crystalline composition described herein and a pharmaceutically acceptable excipient.

In yet other aspects, the disclosure provides methods for treating cancer in a patient in need thereof, the method comprising administering to the patient a composition of described herein. In some embodiments, the cancer is breast cancer, pancreatic cancer, prostate cancer, thyroid cancer, lung cancer, colon cancer, brain cancer, glioma, ovarian cancer, bile duct cancer, cholangiocarcinoma, sarcoma, Hodgkin's lymphoma, lymphoma, renal cancer, tonsil squamous cell carcinoma, or colorectal cancer.

In still further aspects, the disclosure provides methods for preparing a composition described herein.

### BRIEF DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific compositions, methods, devices, and systems disclosed. In addition, the drawings are not necessarily drawn to scale.

FIG. 1 is an X-ray powder diffraction pattern for a composition comprising D-metyrosine.

FIG. 2 is an X-ray powder diffraction pattern for a composition comprising L-metyrosine.

FIG. 3 is an X-ray powder diffraction pattern for a composition comprising about 50% D-metyrosine and about 50% L-metyrosine.

FIG. 4 is an X-ray powder diffraction pattern for a composition comprising about 55% D-metyrosine and about 45% L-metyrosine.

FIG. 5 is an X-ray powder diffraction pattern for a composition comprising about 60% D-metyrosine and about 40% L-metyrosine.

FIG. 6 is an X-ray powder diffraction pattern for a composition comprising about 65% D-metyrosine and about 35% L-metyrosine.

FIG. 7 is an X-ray powder diffraction pattern for a composition comprising about 70% D-metyrosine and about 30% L-metyrosine.

FIG. 8 is an X-ray powder diffraction pattern for a composition comprising about 75% D-metyrosine and about 25% L-metyrosine.

FIG. 9 is an X-ray powder diffraction pattern for a composition comprising about 55% L-metyrosine and about 45% D-metyrosine.

FIG. 10 is an X-ray powder diffraction pattern for a composition comprising about 60% L-metyrosine and about 40% D-metyrosine.

FIG. 11 is an X-ray powder diffraction pattern for a composition comprising about 65% L-metyrosine and about 35% D-metyrosine.

FIG. 12 is an X-ray powder diffraction pattern for a composition comprising about 70% L-metyrosine and about 30% D-metyrosine.

FIG. 13 is an X-ray powder diffraction pattern for a composition comprising about 75% L-metyrosine and about 25% D-metyrosine.

### DETAILED DESCRIPTION OF ILLUSTRATIVE EMBODIMENTS

In the present disclosure the singular forms “a”, “an” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about”. In other cases, the gradations used in a series of values may be used to determine the intended range available to the term “about” for each value. Where present, all ranges are inclusive and combinable. That is, references to values stated in ranges include every value within that range. The modifier “about” also may be considered as disclosing the range defined by the absolute values of the two endpoints. For example, the expression “from about 2 to about 4” also discloses the range “from 2 to 4.” In some embodiments, when used to modify a single number, the term “about” refers to plus or minus 10% of the indicated number and includes the indicated number. For example, “about 10%” indicates a range of 9% to 11%, and “about 1” means from 0.9-1.1. Further, within the context of X-ray diffraction patterns, “about” can also refer to two theta values that vary by about 0.2. By way of example, a two theta value of “about 1.0” is intended to include a two theta value of “0.8 to 1.2”.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

A crystalline form or composition may be referred to herein as being characterized by graphical data “as shown in” a Figure. Such data include, for example, powder X-ray diffractograms (XRPD), among others. As is well-known in the art, the graphical data potentially provides additional technical information to further define the respective solid state form which can not necessarily be described by reference to numerical values or peak positions alone. Thus, the term “substantially as shown in” when referring to graphical data in a Figure herein means a pattern that is not necessarily identical to those depicted herein, but that falls within the limits of experimental error or deviations, when considered by one of ordinary skill in the art. The skilled person would readily be able to compare the graphical data in the Figures herein with graphical data generated for an unknown crystal form and confirm whether the two sets of graphical data are characterizing the same crystal form or two different crystal forms.

A solid, crystalline form may be referred to herein as “polymorphically pure” or as “substantially free of any other form.” As used herein in this context, the expression “substantially free of any other forms” will be understood to mean that the solid form contains about 20% or less, about 10% or less, about 5% or less, about 2% or less, about 1% or less, or 0% of any other forms of the subject compound as measured, for example, by XRPD.

The terms “D-metyrosine,” “D-α-metyrosine,” and “D-α-methyl-tyrosine” as used herein are interchangeably and refer to 2-amino-3-(4-hydroxyphenyl)-2-methylpropanoic acid. D-metyrosine has the following structure:

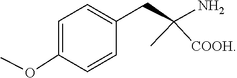

The terms “L-metyrosine,” “L-α-metyrosine,” and “L-α-methyl-tyrosine” as used herein are interchangeably and refer to 2-amino-3-(4-hydroxyphenyl)-2-methylpropanoic acid. L-metyrosine has the following structure:

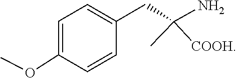

The present disclosure provides crystalline compositions comprising D-metyrosine, L-metyrosine, or a combination thereof. In some embodiments, the disclosure provides crystalline compositions comprising D-metyrosine. In other embodiments, the disclosure provides crystalline compositions comprising L-metyrosine. In further embodiments, the disclosure provides crystalline compositions comprising D-metyrosine and L-metyrosine.

The term “crystalline” as used herein refers to compositions wherein the metyrosine is in the form of crystals having a specific molecular arrangement. It is expected that the crystalline compositions described herein will have one or more advantageous properties. For example, in some embodiments, the crystalline compositions may show advantageous properties, such as, for example, chemical purity, high melting point, substantial lack of solvent (e.g., water) content, little or no weight loss on heating, low hygroscopicity, solubility, flowability, dissolution, and/or bioavailability. In certain embodiments, such properties advantageously facilitate the manufacture, storage and/or formulation of metyrosine.

Crystallinity of the compositions herein can be determined using techniques such as X-ray powder diffraction (XRPD). X-rays will be scattered by electrons in atoms in a substance. Crystalline material will diffract X-rays giving peaks in directions of constructive interference. The directions are determined by the crystal structure, including the size and shape of the unit cell. All diffraction peak °2Theta values disclosed and/or claimed herein refer to Cu Kα-radiation. An amorphous (non-crystalline) material will not give such diffraction peaks. See e.g. Klug, H. P. & Alexander, L. E., X-Ray Diffraction Procedures For Polycrystalline and Amorphous Materials, 1974, John Wiley & Sons.

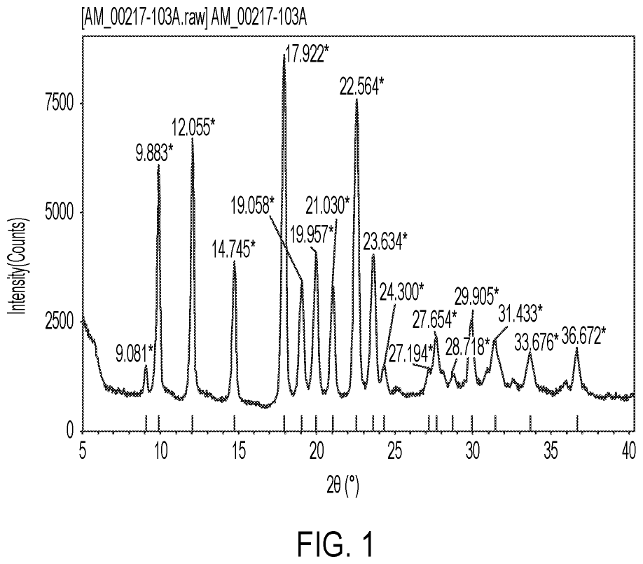

The XRPD of crystalline D-metyrosine shown in FIG. 1 comprises reflection angles (degrees 2 θ±0.2 degrees 2 θ), line spacings (d values), and relative intensities as shown in Table 1 or 1A:

TABLE 1

| 2-Theta ° | d(Å) | H % |
|---|---|---|
| 9.1 | 9.7 | 8.3 |
| 9.9 | 8.9 | 66 |
| 12.1 | 7.3 | 74.9 |
| 14.7 | 6.0 | 40.2 |
| 17.9 | 4.9 | 100 |
| 19.1 | 4.7 | 34.3 |
| 19.9 | 4.4 | 41.9 |
| 21.0 | 4.2 | 31.4 |
| 22.6 | 3.9 | 84.9 |
| 23.6 | 3.8 | 40.4 |
| 24.3 | 3.7 | 9 |
| 27.2 | 3.3 | 6.8 |
| 27.7 | 3.2 | 15.6 |
| 28.7 | 3.1 | 5.7 |
| 29.9 | 3.0 | 20.8 |
| 31.4 | 2.8 | 15.5 |
| 33.7 | 2.7 | 10.6 |
| 36.7 | 2.4 | 13.5 |

TABLE 1A

| 2-Theta ° | d(Å) | H % |
|---|---|---|
| 9.9 | 8.9 | 66 |
| 12.1 | 7.3 | 74.9 |
| 14.7 | 6.0 | 40.2 |
| 17.9 | 4.9 | 100 |
| 19.1 | 4.7 | 34.3 |
| 19.9 | 4.4 | 41.9 |
| 21.0 | 4.2 | 31.4 |
| 22.6 | 3.9 | 84.9 |
| 23.6 | 3.8 | 40.4 |
| 27.7 | 3.2 | 15.6 |
| 29.9 | 3.0 | 20.8 |
| 31.4 | 2.8 | 15.5 |
| 36.7 | 2.4 | 13.5 |

The crystalline D-metyrosine is characterized by an XRPD pattern comprising a peak at one of the angles listed in Table 1 or 1A. In some embodiments, the crystalline D-metyrosine is characterized by an XRPD pattern comprising more than one peak at one of the angles listed in Table 1 or 1A. In other embodiments, the crystalline D-metyrosine is characterized by an XRPD pattern comprising two peaks selected from the angles listed in Table 1 or 1A. In further embodiments, the crystalline D-metyrosine is characterized by an XRPD pattern comprising three peaks selected from the angles listed in Table 1 or 1A. In yet other embodiments, the crystalline D-metyrosine is characterized by an XRPD pattern comprising four peaks selected from the angles listed in Table 1 or 1A. In still further embodiments, the crystalline D-metyrosine is characterized by an XRPD pattern comprising five peaks selected from the angles listed in Table 1 or 1A. In other embodiments, the crystalline D-metyrosine is characterized by an XRPD pattern comprising six peaks selected from the angles listed in Table 1 or 1A. In further embodiments, the crystalline D-metyrosine is characterized by an XRPD pattern comprising seven peaks selected from the angles listed in Table 1 or 1A. In still other embodiments, the crystalline D-metyrosine is characterized by an XRPD pattern comprising eight peaks selected from the angles listed in Table 1 or 1A. In yet further embodiments, the crystalline D-metyrosine is characterized by an XRPD pattern comprising nine peaks selected from the angles listed in Table 1 or 1A. In other embodiments, the crystalline D-metyrosine is characterized by an XRPD pattern comprising ten peaks selected from the angles listed in Table 1 or 1A. In yet further embodiments, the crystalline D-metyrosine is characterized by an XRPD pattern comprising more than ten peaks selected from the angles listed in Table 1 or 1A.

In some embodiments, the crystalline D-metyrosine is characterized by an XRPD pattern comprising three or more peaks at about 9.9, about 12.1, about 14.7, about 17.9, about 19.1, about 19.9, about 21.0, about 22.6, about 23.6, or about 29.9 degrees 2 θ. In other embodiments, the crystalline D-metyrosine is characterized by an XRPD pattern comprising five or more peaks at about 9.9, about 12.1, about 14.7, about 17.9, about 19.1, about 19.9, about 21.0, about 22.6, about 23.6, or about 29.9 degrees 2 θ. In further embodiments, the crystalline D-metyrosine is characterized by an XRPD pattern comprising three or more peaks at about 9.9, about 12.1, about 14.7, about 17.9, about 19.9, about 22.6, and about 23.6 degrees 2 θ. In still other embodiments, the crystalline D-metyrosine is characterized by an XRPD pattern comprising five or more peaks at about 9.9, about 12.1, about 17.9, and about 22.6 degrees 2 θ.

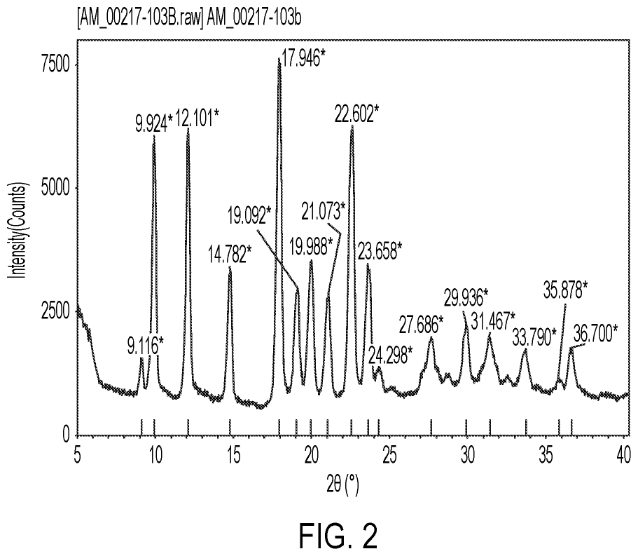

The XRPD of crystalline L-metyrosine shown in FIG. 2 comprises reflection angles (degrees 2-theta 35 0.2 degrees 2-theta), line spacings (d values), and relative intensities as shown in Table 2 or 2A:

TABLE 2

| 2-Theta ° | d(Å) | H % |
|---|---|---|
| 9.1 | 9.7 | 10.6 |
| 9.9 | 8.9 | 75.2 |
| 12.1 | 7.3 | 78.1 |
| 14.8 | 6.0 | 39.1 |
| 17.9 | 4.9 | 100 |
| 19.1 | 4.7 | 32.9 |
| 19.9 | 4.4 | 40.1 |
| 21.1 | 4.2 | 31.3 |
| 22.6 | 3.9 | 77.9 |
| 23.7 | 3.7 | 37.7 |
| 24.3 | 3.7 | 8.2 |
| 27.7 | 3.2 | 15.1 |
| 29.9 | 3.0 | 20.1 |
| 31.5 | 2.8 | 16 |
| 33.8 | 2.6 | 12.2 |
| 35.9 | 2.5 | 4.6 |
| 36.7 | 2.4 | 13.6 |

TABLE 2

| 2-Theta ° | d(Å) | H % |
|---|---|---|
| 9.9 | 8.9 | 75.2 |
| 12.1 | 7.3 | 78.1 |
| 14.8 | 6.0 | 39.1 |
| 17.9 | 4.9 | 100 |
| 19.1 | 4.7 | 32.9 |
| 19.9 | 4.4 | 40.1 |
| 21.1 | 4.2 | 31.3 |
| 22.6 | 3.9 | 77.9 |
| 23.7 | 3.7 | 37.7 |
| 27.7 | 3.2 | 15.1 |
| 29.9 | 3.0 | 20.1 |
| 31.5 | 2.8 | 16 |
| 33.8 | 2.6 | 12.2 |
| 36.7 | 2.4 | 13.6 |

The crystalline L-metyrosine is characterized by an XRPD pattern comprising a peak at one of the angles listed in Table 2 or 2A. In some embodiments, the crystalline L-metyrosine is characterized by an XRPD pattern comprising more than one peak at one of the angles listed in Table 2 or 2A. In other embodiments, the crystalline L-metyrosine is characterized by an XRPD pattern comprising two peaks selected from the angles listed in Table 2 or 2A. In further embodiments, the crystalline L-metyrosine is characterized by an XRPD pattern comprising three peaks selected from the angles listed in Table 2 or 2A. In yet other embodiments, the crystalline L-metyrosine is characterized by an XRPD pattern comprising four peaks selected from the angles listed in Table 2 or 2A. In still further embodiments, the crystalline L-metyrosine is characterized by an XRPD pattern comprising five peaks selected from the angles listed in Table 2 or 2A. In other embodiments, the crystalline L-metyrosine is characterized by an XRPD pattern comprising six peaks selected from the angles listed in Table 2 or 2A. In further embodiments, the crystalline L-metyrosine is characterized by an XRPD pattern comprising seven peaks selected from the angles listed in Table 2 or 2A. In still other embodiments, the crystalline L-metyrosine is characterized by an XRPD pattern comprising eight peaks selected from the angles listed in Table 2 or 2A. In yet further embodiments, the crystalline L-metyrosine is characterized by an XRPD pattern comprising nine peaks selected from the angles listed in Table 2 or 2A. In other embodiments, the crystalline L-metyrosine is characterized by an XRPD pattern comprising ten peaks selected from the angles listed in Table 2 or 2A. In yet further embodiments, the crystalline L-metyrosine is characterized by an XRPD pattern comprising more than ten peaks selected from the angles listed in Table 2 or 2A.

In some embodiments, the crystalline L-metyrosine is characterized by an XRPD pattern comprising three or more peaks at about 9.9, about 12.1, about 14.8, about 17.9, about 19.1, about 19.9, about 21.1, about 22.6, and about 23.7 degrees 2 θ. In other embodiments, the crystalline L-metyrosine is characterized by an XRPD pattern comprising five or more peaks at about 9.9, about 12.1, about 14.8, about 17.9, about 19.1, about 19.9, about 21.1, about 22.6, and about 23.7 degrees 2 θ. In further embodiments, the crystalline L-metyrosine is characterized by an XRPD pattern comprising three or more peaks at about 9.9, about 12.1, about 17.9, about 19.9, and about 22.6 degrees 2 θ. In yet other embodiments, the crystalline L-metyrosine is characterized by an XRPD pattern comprising four or more peaks at about 9.9, about 12.1, about 17.9, about 19.9, and about 22.6 degrees 2 θ.

The crystalline compositions may also comprise varying amounts D-metyrosine and L-metyrosine. In some embodiments, the crystalline compositions are weighted in D-metyrosine, i.e., contain more D-metyrosine than L-metyrosine. In other embodiments, the crystalline compositions in L-metyrosine, i.e., contain more L-metyrosine than D-metyrosine. In some embodiments, the mixture contains L-metyrosine and at least about 10 wt %, based on the weight of the composition, of D-metyrosine. In further embodiments, the mixture contains L-metyrosine and at least about 15 wt %, at least about 20 wt %, at least about 25 wt %, at least about 30 wt %, at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, at least about 80 wt %, at least about 85 wt %, at least about 90 wt %, at least about 95 wt %, or at least about 99 wt %, based on the weight of the composition, of D-metyrosine. In some embodiments, the mixture contains D-metyrosine and at least about 10 wt %, based on the weight of the composition, of L-metyrosine. In other embodiments, the mixture contains D-metyrosine and at least about 15 wt %, at least about 20 wt %, at least about 25 wt %, at least about 30 wt %, at least about 35 wt %, at least about 40 wt %, at least about 45 wt %, at least about 50 wt %, at least about 55 wt %, at least about 60 wt %, at least about 70 wt %, at least about 80 wt %, at least about 90 wt %, or about 100 wt %, based on the weight of the composition, of L-metyrosine.

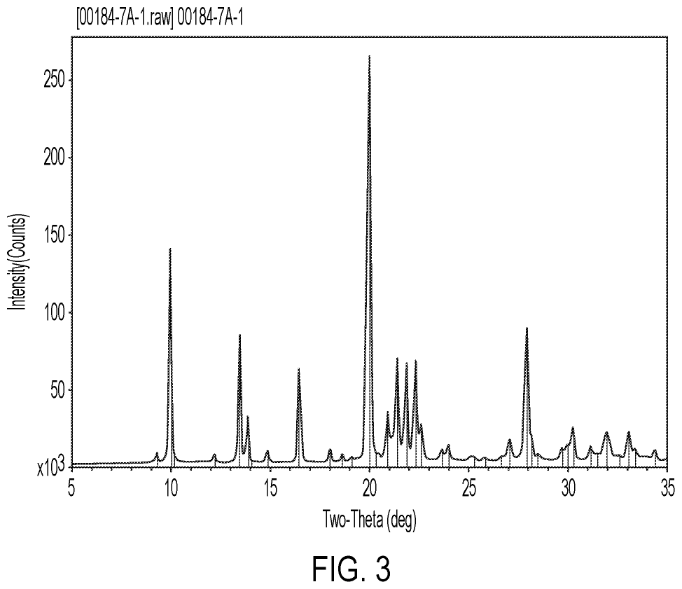

In yet other embodiments, the crystalline compositions contain about 50 wt % of L-metyrosine and about 50 wt % of D-metyrosine, i.e., racemic metyrosine. The XRPD of crystalline racemic metyrosine shown in FIG. 3 comprises reflection angles (degrees 2 θ±0.2 degrees 2 θ), line spacings (d values), and relative intensities as shown in Table 3 or 3A:

TABLE 3

| 2-Theta ° | Height | H % |
|---|---|---|
| 9.3 | 5736 | 2.2 |
| 10.0 | 136832 | 52.9 |
| 10.2 | 4752 | 1.8 |
| 12.2 | 5131 | 2 |
| 13.5 | 81748 | 31.6 |
| 13.9 | 29675 | 11.5 |
| 14.1 | 1055 | 0.4 |
| 14.9 | 6975 | 2.7 |
| 16.5 | 58998 | 22.8 |
| 18.1 | 7662 | 3 |
| 18.7 | 4505 | 1.7 |
| 19.2 | 3104 | 1.2 |
| 20.1 | 258493 | 100 |
| 20.5 | 2464 | 1 |
| 21.0 | 28945 | 11.2 |
| 21.5 | 63562 | 24.6 |
| 22.0 | 60299 | 23.3 |
| 22.4 | 64098 | 24.8 |
| 22.7 | 22401 | 8.7 |
| 23.8 | 6341 | 2.5 |
| 24.1 | 9314 | 3.6 |
| 25.4 | 2898 | 1.1 |
| 26.0 | 1911 | 0.7 |
| 26.7 | 3200 | 1.2 |
| 27.1 | 13354 | 5.2 |
| 28.0 | 83306 | 32.2 |
| 28.2 | 15419 | 6 |
| 28.6 | 3772 | 1.5 |
| 29.8 | 7905 | 3.1 |
| 30.1 | 9659 | 3.7 |
| 30.3 | 20795 | 8 |
| 31.2 | 8825 | 3.4 |
| 31.6 | 3703 | 1.4 |
| 32.0 | 17503 | 6.8 |
| 32.6 | 2817 | 1.1 |
| 33.1 | 16660 | 6.4 |
| 33.4 | 5682 | 2.2 |
| 34.4 | 5309 | 2.1 |

TABLE 3A

| 2-Theta ° | Height | H % |
|---|---|---|
| 10.0 | 136832 | 52.9 |
| 13.5 | 81748 | 31.6 |
| 13.9 | 29675 | 11.5 |
| 16.5 | 58998 | 22.8 |
| 20.1 | 258493 | 100 |
| 21.5 | 63562 | 24.6 |
| 22.0 | 60299 | 23.3 |
| 22.4 | 64098 | 24.8 |
| 28.0 | 83306 | 32.2 |

The crystalline racemic metyrosine is characterized by an XRPD pattern comprising a peak at one of the angles listed in Table 3 or 3A. In some embodiments, crystalline metyrosine is characterized by an XRPD pattern comprising more than one peak at one of the angles listed in Table 3 or 3A. In other embodiments, crystalline racemic metyrosine is characterized by an XRPD pattern comprising two peaks selected from the angles listed in Table 3 or 3A. In further embodiments, crystalline racemic metyrosine is characterized by an XRPD pattern comprising three peaks selected from the angles listed in Table 3 or 3A. In yet other embodiments, crystalline racemic metyrosine is characterized by an XRPD pattern comprising four peaks selected from the angles listed in Table 3 or 3A. In still further embodiments, crystalline racemic metyrosine is characterized by an XRPD pattern comprising five peaks selected from the angles listed in Table 3 or 3A. In other embodiments, the crystalline racemic metyrosine is characterized by an XRPD pattern comprising six peaks selected from the angles listed in Table 3 or 3A. In further embodiments, crystalline racemic metyrosine is characterized by an XRPD pattern comprising seven peaks selected from the angles listed in Table 3 or 3A. In still other embodiments, crystalline racemic metyrosine is characterized by an XRPD pattern comprising eight peaks selected from the angles listed in Table 3 or 3A. In yet further embodiments, crystalline racemic metyrosine is characterized by an XRPD pattern comprising nine peaks selected from the angles listed in Table 3 or 3A. In other embodiments, crystalline racemic metyrosine is characterized by an XRPD pattern comprising ten peaks selected from the angles listed in Table 3 or 3A. In yet further embodiments, crystalline racemic metyrosine is characterized by an XRPD pattern comprising more than ten peaks selected from the angles listed in Table 3 or 3A.

In some embodiments, crystalline racemic metyrosine is characterized by an XRPD pattern comprising three or more peaks at about 10.0, about 13.5, about 16.5, about 20.1, about 21.5, about 22.0, about 22.4, and about 28.0 degrees 2 θ. In other embodiments, crystalline racemic metyrosine is characterized by an XRPD pattern comprising five or more peaks at about 10.0, about 13.5, about 16.5, about 20.1, about 21.5, about 22.0, about 22.4, and about 28.0 degrees 2 θ. In further embodiments, crystalline racemic metyrosine is characterized by an XRPD pattern comprising three or more peaks at about 10.0, about 13.5, about 20.1, and about 28.0 degrees 2 θ. In still other embodiments, crystalline racemic metyrosine is characterized by an XRPD pattern comprising peaks at about 10.0, about 13.5, about 20.1, and about 28.0 degrees 2 θ.

### Compositions Weighted in D-Metyrosine

The disclosure provides crystalline compositions comprising about 55% to about 95% by weight of D-metyrosine and about 5% to about 45% by weight of L-metyrosine, based on the total weight of the crystalline composition. In some aspects, the crystalline composition comprises about 55% by weight of D-metyrosine and about 45% by weight of L-metyrosine. In other aspects, the crystalline composition comprises about 60% by weight of D-metyrosine and about 40% by weight of L-metyrosine. In further aspects, the crystalline composition comprises about 65% by weight of D-metyrosine and about 35% by weight of L-metyrosine. In still other aspects, the crystalline composition comprises about 70% by weight of D-metyrosine and about 30% by weight of L-metyrosine. In other aspects, the crystalline composition comprises about 75% by weight of D-metyrosine and about 25% by weight of L-metyrosine. In yet further aspects, the crystalline composition comprises about 80% by weight of D-metyrosine and about 20% by weight of L-metyrosine. In other aspects, the crystalline composition comprises about 85% by weight of D-metyrosine and about 15% by weight of L-metyrosine. In further aspects, the crystalline composition comprises about 90% by weight of D-metyrosine and about 10% by weight of L-metyrosine. In yet other aspects, the crystalline composition comprises about 95% by weight of D-metyrosine and about 5% by weight of L-metyrosine. In still other aspects, the crystalline composition comprises about 55 to about 75% by weight of D-metyrosine and about 25% to about 45% by weight of L-metyrosine.

Such compositions are characterized by a powder X-ray diffraction pattern comprising at least three peaks selected from about 10.2, about 13.5, about 20.1, about 21.5, about 22.5, or about 28.0 degrees 2 θ. In some embodiments, the XRPD comprises at least four peaks selected from about 10.2, about 13.5, about 20.1, about 21.5, about 22.5, or about 28.0 degrees 2 θ. In other embodiments, the XRPD pattern comprises at least five peaks selected from about 10.2, about 13.5, about 20.1, about 21.5, about 22.5, or about 28.0 degrees 2 θ.

The XRPD pattern may comprise one or more peaks in addition to those described above. In some embodiments, the XRPD pattern further comprises one or more peaks at about 16.6, about 22.0, or about 32.6 degrees 2 θ. In other embodiments, the XRPD pattern further comprises one more peaks at about 13.9, about 14.9, about 22.7, about 23.8, or about 30.1 degrees 2 θ. In further embodiments, the SRPD pattern further comprises one more peaks at about 12.2, about 13.9, about 14.9, about 18.1, about 23.8, or about 30.1 degrees 2 θ. In yet other embodiments, the XRPD pattern further comprises one more peaks at about 12.3, about 13.9, about 15.0, about 18.1, about 19.2, about 21.1, about 21.3, about 22.8, about 23.8, about 30.1, or about 31.6 degrees 2 θ.

### Compositions Weighted in L-Metyrosine

The disclosure provides crystalline compositions comprising about 55% to about 95% by weight of L-metyrosine and about 5% to about 45% by weight of D-metyrosine, based on the total weight of the crystalline composition. In some embodiments, the disclosure provides crystalline compositions comprising about 55 to about 75% by weight of L-metyrosine and about 25% to about 45% by weight of D-metyrosine. In other embodiments, the disclosure provides crystalline compositions comprising about 55% by weight of L-metyrosine and about 45% by weight of D-metyrosine. In further embodiments, the disclosure provides crystalline compositions comprising about 60% by weight of L-metyrosine and about 40% by weight of D-metyrosine. In still other embodiments, the disclosure provides crystalline compositions comprising about 65% by weight of L-metyrosine and about 35% by weight of D-metyrosine. In yet further embodiments, the disclosure provides crystalline compositions comprising about 70% by weight of L-metyrosine and about 30% by weight of D-metyrosine. In other embodiments, the disclosure provides crystalline compositions comprising about 80% by weight of L-metyrosine and about 20% by weight of D-metyrosine. In further embodiments, the disclosure provides crystalline compositions comprising about 85% by weight of L-metyrosine and about 15% by weight of D-metyrosine. In still other embodiments, the disclosure provides crystalline compositions comprising about 90% by weight of L-metyrosine and about 10% by weight of D-metyrosine. In yet other embodiments, the disclosure provides crystalline compositions comprising about 95% by weight of L-metyrosine and about 5% by weight of D-metyrosine.

In some aspects, the crystalline compositions comprise about 10% by weight of D-metyrosine and about 90% by weight of L-metyrosine. In some aspects, the crystalline compositions contains about 15% by weight of D-metyrosine and about 85% by weight of L-metyrosine. In further aspects, the crystalline compositions contains about 20% by weight of D-metyrosine and about 80% by weight of L-metyrosine. In other aspects, the crystalline compositions contains about 25% by weight of D-metyrosine and about 75% by weight of L-metyrosine. In still further aspects, the crystalline compositions contains about 30% by weight of D-metyrosine and about 70% by weight of L-metyrosine. In other aspects, the crystalline compositions contains about 35% by weight of D-metyrosine and about 65% by weight of L-metyrosine. In further aspects, the crystalline compositions contains about 40% by weight of D-metyrosine and about 60% by weight of L-metyrosine. In still other aspects, the crystalline compositions contains about 45% by weight of D-metyrosine and about 55% by weight of L-metyrosine.

Such compositions are characterized by a powder X-ray diffraction pattern comprising at least three peaks selected from about 9.9, about 13.4, about 16.4, about 20.0, or about 21.9 degrees 2 θ. In other embodiments, the XRPD pattern contains at least four peaks selected from about 9.9, about 13.4, about 16.4, about 20.0, or about 21.9. In further embodiments, the XRPD pattern contains at least five peaks selected from about 9.9, about 13.4, about 16.4, about 20.0, or about 21.9 degrees 2 θ.

The XRPD pattern may comprise one or more peaks in addition to those described above. In some embodiments, the XRPD pattern further comprises one or more peaks at about 13.8, about 21.0, about 21.4, about 22.4, about 27.9, or about 32.6. In other embodiments, the XRPD pattern further comprises one or more peaks at about 9.2, about 30.2, about 31.9, about 33.1, or about 37.8 degrees 2 θ. In further embodiments, the XRPD further comprises one or more peaks at about 14.8 or about 22.7 degrees 2 θ. In yet other embodiments, the XRPD pattern further comprises one or more peaks at about 21.1, about 14.8, about 17.9, about 22.7, about 23.7, or about 29.9 degrees 2 θ. In still further embodiments, the XRPD pattern further comprises one or more peaks at about 12.1, about 14.8, about 18.0, about 19.1, about 22.7, or about 23.7 degrees 2 θ. In other embodiments, the XRPD pattern further comprises one or more peaks at about 12.1, about 14.8, about 18.0, about 19.1, about 22.7, about 23.7, about 30.0, about 31.5, or about 31.9 degrees 2 θ.

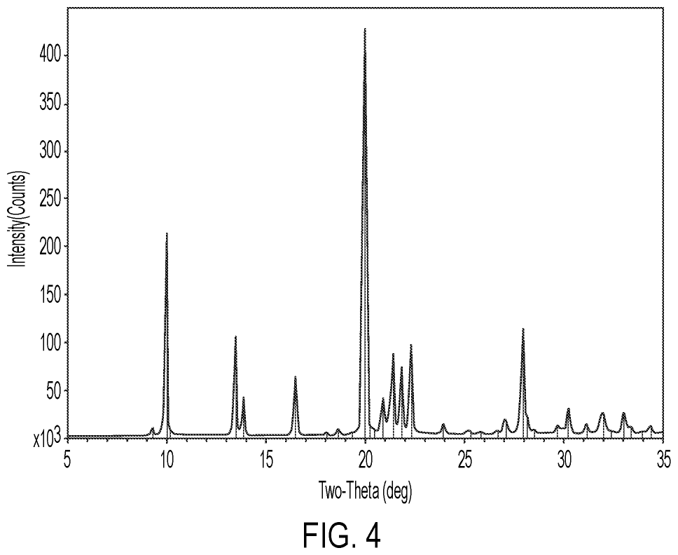

The XRPD data for a crystalline composition comprising about 55% by weight of D-metyrosine and about 45% by weight of L-metyrosine shown in FIG. 4 comprises reflection angles (degrees 2 θ±0.2 degrees 2 θ), line spacings (d values), and relative intensities as shown in Table 4 or 4A:

TABLE 4

| 2-Theta ° | d(Å) | H % |
|---|---|---|
| 9.3 | 9.5 | 1.7 |
| 10.0 | 8.8 | 49.3 |
| 10.2 | 8.7 | 1.5 |
| 13.5 | 6.5 | 24.1 |
| 13.9 | 6.4 | 9.2 |
| 14.1 | 6.3 | 0.4 |
| 16.6 | 5.3 | 14.4 |
| 18.1 | 4.9 | 0.4 |
| 18.7 | 4.7 | 1.5 |
| 19.5 | 4.6 | 0.7 |
| 20.1 | 4.4 | 100 |
| 20.3 | 4.4 | 3.2 |
| 20.6 | 4.3 | 1.5 |
| 21.0 | 4.2 | 8.8 |
| 21.5 | 4.1 | 19.8 |
| 22.0 | 4.0 | 16.4 |
| 22.5 | 3.9 | 22 |
| 24.1 | 3.7 | 2.4 |
| 25.4 | 3.5 | 0.7 |
| 26.0 | 3.4 | 0.5 |
| 26.7 | 3.3 | 0.7 |
| 27.2 | 3.3 | 3.5 |
| 28.0 | 3.2 | 25.5 |
| 28.2 | 3.1 | 4.5 |
| 28.6 | 3.1 | 0.9 |
| 29.8 | 3.0 | 2.1 |
| 30.3 | 2.9 | 6.1 |
| 31.2 | 2.9 | 2.2 |
| 32.1 | 2.8 | 4.8 |
| 32.5 | 2.8 | 0.5 |
| 33.1 | 2.7 | 5.1 |
| 33.4 | 2.7 | 1.7 |
| 34.0 | 2.6 | 0.4 |
| 34.4 | 2.6 | 1.7 |

TABLE 4A

| 2-Theta ° | d(Å) | H % |
|---|---|---|
| 10.0 | 8.8 | 49.3 |
| 13.5 | 6.5 | 24.1 |
| 16.6 | 5.3 | 14.4 |
| 20.1 | 4.4 | 100 |
| 21.5 | 4.1 | 19.8 |
| 22.0 | 4.0 | 16.4 |
| 22.5 | 3.9 | 22 |
| 28.0 | 3.2 | 25.5 |

The crystalline composition comprising about 55% by weight of D-metyrosine and about 45% by weight of L-metyrosine is characterized by an XRPD pattern comprising a peak at one of the angles listed in Table 4 or 4A. In some embodiments, the crystalline composition comprising about 55% by weight of D-metyrosine and about 45% by weight of L-metyrosine is characterized by an XRPD pattern comprising more than one peak at one of the angles listed in Table 4 or 4A. In other embodiments, the crystalline composition comprising about 55% by weight of D-metyrosine and about 45% by weight of L-metyrosine is characterized by an XRPD pattern comprising two peaks selected from the angles listed in Table 4 or 4A. In further embodiments, the crystalline composition comprising about 55% by weight of D-metyrosine and about 45% by weight of L-metyrosine is characterized by an XRPD pattern comprising three peaks selected from the angles listed in Table 4 or 4A. In yet other embodiments, the crystalline composition comprising about 55% by weight of D-metyrosine and about 45% by weight of L-metyrosine is characterized by an XRPD pattern comprising four peaks selected from the angles listed in Table 4 or 4A. In still further embodiments, the crystalline composition comprising about 55% by weight of D-metyrosine and about 45% by weight of L-metyrosine is characterized by an XRPD pattern comprising five peaks selected from the angles listed in Table 4 or 4A. In other embodiments, the crystalline composition comprising about 55% by weight of D-metyrosine and about 45% by weight of L-metyrosine is characterized by an XRPD pattern comprising six peaks selected from the angles listed in Table 4 or 4A. In further embodiments, the crystalline composition comprising about 55% by weight of D-metyrosine and about 45% by weight of L-metyrosine is characterized by an XRPD pattern comprising seven peaks selected from the angles listed in Table 4 or 4A. In still other embodiments, the crystalline composition comprising about 55% by weight of D-metyrosine and about 45% by weight of L-metyrosine is characterized by an XRPD pattern comprising eight peaks selected from the angles listed in Table 4 or 4A. In yet further embodiments, the crystalline composition comprising about 55% by weight of D-metyrosine and about 45% by weight of L-metyrosine is characterized by an XRPD pattern comprising nine peaks selected from the angles listed in Table 4. In other embodiments, the crystalline composition comprising about 55% by weight of D-metyrosine and about 45% by weight of L-metyrosine is characterized by an XRPD pattern comprising ten peaks selected from the angles listed in Table 4. In yet further embodiments, the crystalline composition comprising about 55% by weight of D-metyrosine and about 45% by weight of L-metyrosine is characterized by an XRPD pattern comprising more than ten peaks selected from the angles listed in Table 4.

In some embodiments, the crystalline composition comprising about 55% by weight of D-metyrosine and about 45% by weight of L-metyrosine is characterized by an XRPD pattern comprising three or more peaks at about 10.0, about 13.5, about 20.1, about 22.5, and about 28.0 degrees 2 θ. In other embodiments, the crystalline composition comprising about 55% by weight of D-metyrosine and about 45% by weight of L-metyrosine is characterized by an XRPD pattern comprising four or more peaks at about 10.0, about 13.5, about 20.1, about 22.5, and about 28.0 degrees 2 θ. In further embodiments, the crystalline composition comprising about 55% by weight of D-metyrosine and about 45% by weight of L-metyrosine is characterized by an XRPD pattern comprising peaks at about 10.0, about 13.5, about 20.1, about 22.5, and about 28.0 degrees 2 θ.

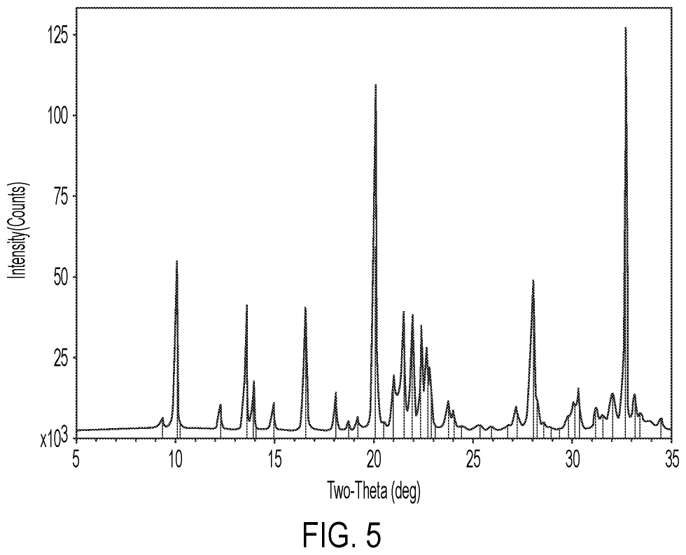

The XRPD of the crystalline composition comprising about 60% by weight of D-metyrosine and about 40% by weight of L-metyrosine shown in FIG. 5 comprises reflection angles (degrees 2 θ±0.2 degrees 2 θ), line spacings (d values), and relative intensities as shown in Table 5 or 5A:

TABLE 5

| 2-Theta ° | d(Å) | H % |
|---|---|---|
| 9.4 | 9.4 | 2.3 |
| 10.1 | 8.8 | 41.3 |
| 10.2 | 8.7 | 1.7 |
| 12.2 | 7.2 | 5.9 |
| 13.5 | 6.5 | 30.8 |
| 13.9 | 6.3 | 12 |
| 14.1 | 6.3 | 0.4 |
| 14.9 | 5.9 | 6.7 |
| 16.6 | 5.3 | 30.5 |
| 18.1 | 4.9 | 9.3 |
| 18.8 | 4.7 | 2 |
| 19.2 | 4.6 | 2.7 |
| 20.1 | 4.4 | 84.4 |
| 20.6 | 4.3 | 1.2 |
| 21.1 | 4.2 | 12.4 |
| 21.6 | 4.1 | 28.3 |
| 22.0 | 4.0 | 27.4 |
| 22.5 | 3.9 | 25 |
| 22.8 | 3.9 | 19.5 |
| 22.9 | 3.9 | 14.8 |
| 23.2 | 3.8 | 0.4 |
| 23.8 | 3.7 | 6.5 |
| 24.2 | 3.7 | 4.7 |
| 24.5 | 3.6 | 0.7 |
| 25.4 | 3.5 | 1.3 |
| 26.0 | 3.4 | 0.9 |
| 26.8 | 3.3 | 1.4 |
| 27.2 | 3.3 | 6 |
| 28.0 | 3.2 | 37 |
| 28.3 | 3.1 | 7.9 |
| 28.6 | 3.1 | 2 |
| 28.9 | 3.1 | 0.8 |
| 29.4 | 3.0 | 0.7 |
| 29.8 | 3.0 | 3.8 |
| 30.1 | 3.0 | 7.2 |
| 30.4 | 2.9 | 10.1 |
| 31.2 | 2.9 | 5.7 |
| 31.6 | 2.8 | 3.8 |
| 32.0 | 2.8 | 9.1 |
| 32.7 | 2.7 | 100 |
| 33.2 | 2.7 | 8.8 |
| 33.5 | 2.7 | 4.5 |
| 34.5 | 2.6 | 3.2 |

TABLE 5A

| 2-Theta ° | d(Å) | H % |
|---|---|---|
| 10.1 | 8.8 | 41.3 |
| 13.5 | 6.5 | 30.8 |
| 13.9 | 6.3 | 12 |
| 16.6 | 5.3 | 30.5 |
| 20.1 | 4.4 | 84.4 |
| 21.1 | 4.2 | 12.4 |
| 21.6 | 4.1 | 28.3 |
| 22.0 | 4.0 | 27.4 |
| 22.5 | 3.9 | 25 |
| 22.8 | 3.9 | 19.5 |
| 22.9 | 3.9 | 14.8 |
| 28.0 | 3.2 | 37 |
| 32.7 | 2.7 | 100 |

The crystalline composition comprising about 60% by weight of D-metyrosine and about 40% by weight of L-metyrosine is characterized by an XRPD pattern comprising a peak at one of the angles listed in Table 5 or 5A. In some embodiments, the crystalline composition comprising about 60% by weight of D-metyrosine and about 40% by weight of L-metyrosine is characterized by an XRPD pattern comprising more than one peak at one of the angles listed in Table 5 or 5A. In other embodiments, the crystalline composition comprising about 60% by weight of D-metyrosine and about 40% by weight of L-metyrosine is characterized by an XRPD pattern comprising two peaks selected from the angles listed in Table 5 or 5A. In further embodiments, the crystalline composition comprising about 60% by weight of D-metyrosine and about 40% by weight of L-metyrosine is characterized by an XRPD pattern comprising three peaks selected from the angles listed in Table 5 or 5A. In yet other embodiments, the crystalline composition comprising about 60% by weight of D-metyrosine and about 40% by weight of L-metyrosine is characterized by an XRPD pattern comprising four peaks selected from the angles listed in Table 5 or 5A. In still further embodiments, the crystalline composition comprising about 60% by weight of D-metyrosine and about 40% by weight of L-metyrosine characterized by an XRPD pattern comprising five peaks selected from the angles listed in Table 5 or 5A. In other embodiments, the crystalline composition comprising about 60% by weight of D-metyrosine and about 40% by weight of L-metyrosine is characterized by an XRPD pattern comprising six peaks selected from the angles listed in Table 5 or 5A. In further embodiments, the crystalline composition comprising about 60% by weight of D-metyrosine and about 40% by weight of L-metyrosine is characterized by an XRPD pattern comprising seven peaks selected from the angles listed in Table 5 or 5A. In still other embodiments, the crystalline composition comprising about 60% by weight of D-metyrosine and about 40% by weight of L-metyrosine is characterized by an XRPD pattern comprising eight peaks selected from the angles listed in Table 5 or 5A. In yet further embodiments, the crystalline composition comprising about 60% by weight of D-metyrosine and about 40% by weight of L-metyrosine is characterized by an XRPD pattern comprising nine peaks selected from the angles listed in Table 5 or 5A. In other embodiments, the crystalline composition comprising about 60% by weight of D-metyrosine and about 40% by weight of L-metyrosine is characterized by an XRPD pattern comprising ten peaks selected from the angles listed in Table 5 or 5A. In yet further embodiments, the crystalline composition comprising about 60% by weight of D-metyrosine and about 40% by weight of L-metyrosine is characterized by an XRPD pattern comprising more than ten peaks selected from the angles listed in Table 5 or 5A.

In some embodiments, the crystalline composition comprising about 60% by weight of D-metyrosine and about 40% by weight of L-metyrosine is characterized by an XRPD pattern comprising three or more peaks at about 10.1, about 13.5, about 16.6, about 20.1, about 21.6, about 22.0, about 22.5, about 28.0, and about 32.7 degrees 2 θ. In other embodiments, the crystalline composition comprising about 60% by weight of D-metyrosine and about 40% by weight of L-metyrosine is characterized by an XRPD pattern comprising five or more peaks at about 10.1, about 13.5, about 16.6, about 20.1, about 21.6, about 22.0, about 22.5, about 28.0, and about 32.7 degrees 2 θ. In further embodiments, the crystalline composition comprising about 60% by weight of D-metyrosine and about 40% by weight of L-metyrosine is characterized by an XRPD pattern comprising three or more peaks at about 10.1, about 13.5, about 16.6, about 20.1, about 28.0, and about 32.7 degrees 2 θ. In still other embodiments, the crystalline composition comprising about 60% by weight of D-metyrosine and about 40% by weight of L-metyrosine is characterized by an XRPD pattern comprising five or more peaks at about 10.1, about 13.5, about 16.6, about 20.1, about 28.0, and about 32.7 degrees 2 θ.

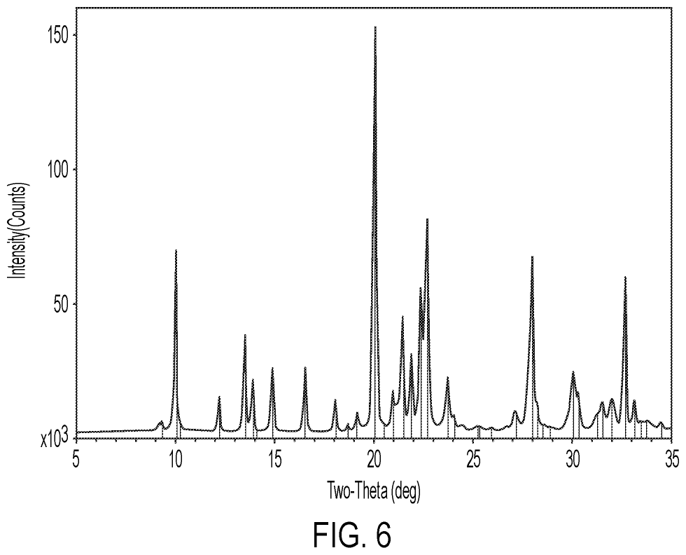

The XRPD of the crystalline composition comprising about 65% by weight of D-metyrosine and about 35% by weight of L-metyrosine shown in FIG. 6 comprises reflection angles (degrees 2 θ±0.2 degrees 2 θ), line spacings (d values), and relative intensities as shown in Table 6 or 6A:

TABLE 6

| 2-Theta ° | d(Å) | H % |
|---|---|---|
| 9.3 | 9.5 | 1.8 |
| 10.4 | 8.8 | 44.6 |
| 10.2 | 8.7 | 2.2 |
| 12.2 | 7.2 | 8.5 |
| 13.5 | 6.5 | 24.1 |
| 13.9 | 6.4 | 12.8 |
| 14.1 | 6.3 | 0.4 |
| 14.9 | 5.9 | 15.7 |
| 16.6 | 5.3 | 16.1 |
| 18.1 | 4.9 | 7.9 |
| 18.7 | 4.7 | 1.5 |
| 19.2 | 4.6 | 4.6 |
| 20.1 | 4.4 | 100 |
| 20.5 | 4.3 | 1 |
| 21.0 | 4.2 | 8.9 |
| 21.5 | 4.1 | 27 |
| 22.0 | 4.0 | 18.1 |
| 22.5 | 4.0 | 34.7 |
| 22.7 | 3.9 | 51.5 |
| 23.8 | 3.7 | 12.3 |
| 24.1 | 3.7 | 3 |
| 25.3 | 3.5 | 0.8 |
| 25.4 | 3.5 | 0.8 |
| 26.0 | 3.4 | 0.6 |
| 27.2 | 3.3 | 4.9 |
| 28.0 | 3.2 | 43.2 |
| 28.2 | 3.2 | 6.9 |
| 28.6 | 3.1 | 1.6 |
| 28.9 | 3.1 | 0.8 |
| 30.1 | 3.0 | 14.2 |
| 30.3 | 2.9 | 9.1 |
| 31.3 | 2.9 | 3.5 |
| 31.5 | 2.8 | 6.5 |
| 32.0 | 2.8 | 7.2 |
| 32.7 | 2.7 | 37.6 |
| 33.1 | 2.7 | 6.8 |
| 33.5 | 2.7 | 1.9 |
| 33.8 | 2.7 | 2.2 |
| 34.5 | 2.6 | 1.6 |

TABLE 6A

| 2-Theta ° | d(Å) | H % |
|---|---|---|
| 10.4 | 8.8 | 44.6 |
| 13.5 | 6.5 | 24.1 |
| 13.9 | 6.4 | 12.8 |
| 14.9 | 5.9 | 15.7 |
| 16.6 | 5.3 | 16.1 |
| 20.1 | 4.4 | 100 |
| 21.5 | 4.1 | 27 |
| 22.0 | 4.0 | 18.1 |
| 22.5 | 4.0 | 34.7 |
| 22.7 | 3.9 | 51.5 |
| 23.8 | 3.7 | 12.3 |
| 28.0 | 3.2 | 43.2 |
| 30.1 | 3.0 | 14.2 |
| 32.7 | 2.7 | 37.6 |

The crystalline composition comprising about 65% by weight of D-metyrosine and about 35% by weight of L-metyrosine is characterized by an XRPD pattern comprising a peak at one of the angles listed in Table 6 or 6A. In some embodiments, the crystalline composition comprising about 65% by weight of D-metyrosine and about 35% by weight of L-metyrosine is characterized by an XRPD pattern comprising more than one peak at one of the angles listed in Table 6 or 6A. In other embodiments, the crystalline composition comprising about 65% by weight of D-metyrosine and about 35% by weight of L-metyrosine is characterized by an XRPD pattern comprising two peaks selected from the angles listed in Table 6 or 6A. In further embodiments, the crystalline composition comprising about 65% by weight of D-metyrosine and about 35% by weight of L-metyrosine is characterized by an XRPD pattern comprising three peaks selected from the angles listed in Table 6 or 6A. In yet other embodiments, the crystalline composition comprising about 65% by weight of D-metyrosine and about 35% by weight of L-metyrosine is characterized by an XRPD pattern comprising four peaks selected from the angles listed in Table 6 or 6A. In still further embodiments, the crystalline composition comprising about 65% by weight of D-metyrosine and about 35% by weight of L-metyrosine is characterized by an XRPD pattern comprising five peaks selected from the angles listed in Table 6 or 6A. In other embodiments, the crystalline composition comprising about 65% by weight of D-metyrosine and about 35% by weight of L-metyrosine is characterized by an XRPD pattern comprising six peaks selected from the angles listed in Table 6 or 6A. In further embodiments, the crystalline composition comprising about 65% by weight of D-metyrosine and about 35% by weight of L-metyrosine is characterized by an XRPD pattern comprising seven peaks selected from the angles listed in Table 6 or 6A. In still other embodiments, the crystalline composition comprising about 65% by weight of D-metyrosine and about 35% by weight of L-metyrosine is characterized by an XRPD pattern comprising eight peaks selected from the angles listed in Table 6 or 6A. In yet further embodiments, the crystalline composition comprising about 65% by weight of D-metyrosine and about 35% by weight of L-metyrosine is characterized by an XRPD pattern comprising nine peaks selected from the angles listed in Table 6 or 6A. In other embodiments, the crystalline composition comprising about 65% by weight of D-metyrosine and about 35% by weight of L-metyrosine is characterized by an XRPD pattern comprising ten peaks selected from the angles listed in Table 6 or 6A. In yet further embodiments, the crystalline composition comprising about 65% by weight of D-metyrosine and about 35% by weight of L-metyrosine is characterized by an XRPD pattern comprising more than ten peaks selected from the angles listed in Table 6 or 6A.

In some embodiments, the crystalline composition comprising about 65% by weight of D-metyrosine and about 35% by weight of L-metyrosine is characterized by an XRPD pattern comprising three or more peaks at about 10.5, about 13.5, about 20.1, about 21.5, about 22.5, about 22.8, about 28.0, and about 32.7 degrees 2 θ. In other embodiments, the crystalline composition comprising about 65% by weight of D-metyrosine and about 35% by weight of L-metyrosine is characterized by an XRPD pattern comprising five or more peaks at 10.5, 13.5, 20.1, 21.5, 22.5, 22.8, 28.0, and 32.7 degrees 2 θ. In further embodiments, the crystalline composition comprising about 65% by weight of D-metyrosine and about 35% by weight of L-metyrosine is characterized by an XRPD pattern comprising three or more peaks at about 10.5, about 20.1, about 22.5, about 22.8, about 28.0, and about 32.7 degrees 2 θ. In further embodiments, the crystalline composition comprising about 65% by weight of D-metyrosine and about 35% by weight of L-metyrosine is characterized by an XRPD pattern comprising five or more peaks at about 10.5, about 20.1, about 22.5, about 22.8, about 28.0, and about 32.7 degrees 2 θ.

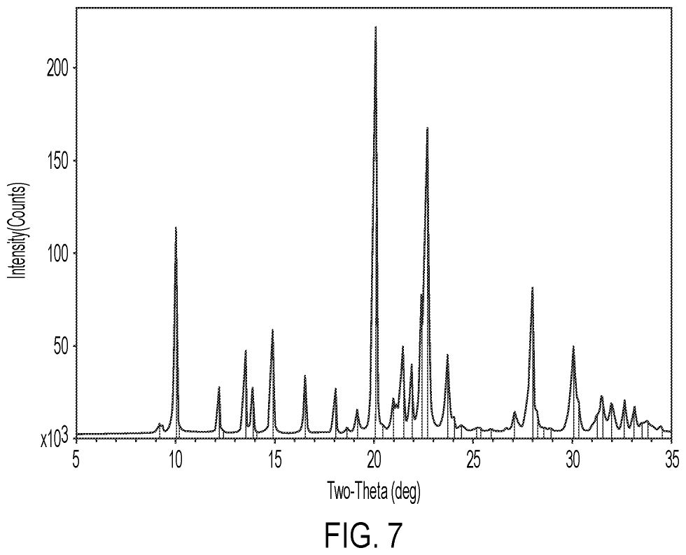

The XRPD of the crystalline composition comprising about 70% by weight of D-metyrosine and about 30% by weight of L-metyrosine shown in FIG. 7 comprises reflection angles (degrees 2 θ±0.2 degrees 2 θ), line spacings (d values), and relative intensities as shown in Table 7 or 7A:

TABLE 7

| 2-Theta ° | d(Å) | H % |
|---|---|---|
| 9.2 | 9.6 | 2.1 |
| 10.0 | 8.8 | 50.5 |
| 10.2 | 8.7 | 1.8 |
| 12.2 | 7.2 | 11.6 |
| 12.4 | 7.1 | 0.4 |
| 13.5 | 6.6 | 20.5 |
| 13.9 | 6.4 | 11.1 |
| 14.1 | 6.3 | 0.2 |
| 14.9 | 5.9 | 25.8 |
| 16.5 | 5.4 | 14.5 |
| 18.1 | 4.9 | 10.9 |
| 18.7 | 4.7 | 1.2 |
| 19.2 | 4.6 | 5.8 |
| 20.1 | 4.4 | 100 |
| 20.4 | 4.3 | 1.1 |
| 21.0 | 4.2 | 7.3 |
| 21.5 | 4.1 | 20.4 |
| 22.0 | 4.0 | 16 |
| 22.4 | 4.0 | 33.4 |
| 22.7 | 3.9 | 74.6 |
| 23.8 | 3.7 | 18.8 |
| 24.1 | 3.7 | 3.4 |
| 24.5 | 3.6 | 1.3 |
| 25.2 | 3.5 | 0.8 |
| 25.4 | 3.5 | 0.8 |
| 26.0 | 3.4 | 0.5 |
| 27.2 | 3.3 | 5 |
| 28.0 | 3.2 | 34.8 |
| 28.2 | 3.2 | 5.4 |
| 28.6 | 3.1 | 1 |
| 28.9 | 3.1 | 0.6 |
| 30.1 | 3.0 | 20.8 |
| 30.3 | 2.9 | 7.7 |
| 31.3 | 2.9 | 3.6 |
| 31.5 | 2.8 | 8.6 |
| 32.0 | 2.8 | 6.7 |
| 32.6 | 2.7 | 7.5 |
| 33.1 | 2.7 | 5.9 |
| 33.5 | 2.7 | 1.8 |
| 33.7 | 2.7 | 2.9 |
| 34.4 | 2.6 | 1.4 |

TABLE 7A

| 2-Theta ° | d(Å) | H % |
|---|---|---|
| 10.0 | 8.8 | 50.5 |
| 12.2 | 7.2 | 11.6 |
| 13.5 | 6.6 | 20.5 |
| 13.9 | 6.4 | 11.1 |
| 14.9 | 5.9 | 25.8 |
| 16.5 | 5.4 | 14.5 |
| 20.1 | 4.4 | 100 |
| 21.5 | 4.1 | 20.4 |
| 22.0 | 4.0 | 16 |
| 22.4 | 4.0 | 33.4 |
| 22.7 | 3.9 | 74.6 |
| 23.8 | 3.7 | 18.8 |
| 28.0 | 3.2 | 34.8 |
| 30.1 | 3.0 | 20.8 |

The crystalline composition comprising about 70% by weight of D-metyrosine and about 30% by weight of L-metyrosine is characterized by an XRPD pattern comprising a peak at one of the angles listed in Table 7 or 7A. In some embodiments, the crystalline composition comprising about 70% by weight of D-metyrosine and about 30% by weight of L-metyrosine is characterized by an XRPD pattern comprising more than one peak at one of the angles listed in Table 7 or 7A. In other embodiments, the crystalline composition comprising about 70% by weight of D-metyrosine and about 30% by weight of L-metyrosine is characterized by an XRPD pattern comprising two peaks selected from the angles listed in Table 7 or 7A. In further embodiments, the crystalline composition comprising about 70% by weight of D-metyrosine and about 30% by weight of L-metyrosine is characterized by an XRPD pattern comprising three peaks selected from the angles listed in Table 7 or 7A. In yet other embodiments, the crystalline composition comprising about 70% by weight of D-metyrosine and about 30% by weight of L-metyrosine is characterized by an XRPD pattern comprising four peaks selected from the angles listed in Table 7 or 7A. In still further embodiments, the crystalline composition comprising about 70% by weight of D-metyrosine and about 30% by weight of L-metyrosine is characterized by an XRPD pattern comprising five peaks selected from the angles listed in Table 7 or 7A. In other embodiments, the crystalline composition comprising about 70% by weight of D-metyrosine and about 30% by weight of L-metyrosine is characterized by an XRPD pattern comprising six peaks selected from the angles listed in Table 7 or 7A. In further embodiments, the crystalline composition comprising about 70% by weight of D-metyrosine and about 30% by weight of L-metyrosine is characterized by an XRPD pattern comprising seven peaks selected from the angles listed in Table 7 or 7A. In still other embodiments, the crystalline composition comprising about 70% by weight of D-metyrosine and about 30% by weight of L-metyrosine is characterized by an XRPD pattern comprising eight peaks selected from the angles listed in Table 7 or 7A. In yet further embodiments, the crystalline composition comprising about 70% by weight of D-metyrosine and about 30% by weight of L-metyrosine is characterized by an XRPD pattern comprising nine peaks selected from the angles listed in Table 7 or 7A. In other embodiments, the crystalline composition comprising about 70% by weight of D-metyrosine and about 30% by weight of L-metyrosine is characterized by an XRPD pattern comprising ten peaks selected from the angles listed in Table 7 or 7A. In yet further embodiments, the crystalline composition comprising about 70% by weight of D-metyrosine and about 30% by weight of L-metyrosine is characterized by an XRPD pattern comprising more than ten peaks selected from the angles listed in Table 7 or 7A.

In some embodiments, the crystalline composition comprising about 70% by weight of D-metyrosine and about 30% by weight of L-metyrosine is characterized by an XRPD pattern comprising three or more peaks at 10.0, 14.9, 20.1, 21.5, 22.5, 22.7, 28.0, and 30.1 degrees 2 θ. In other embodiments, the crystalline composition comprising about 70% by weight of D-metyrosine and about 30% by weight of L-metyrosine is characterized by an XRPD pattern comprising five or more peaks at about 10.0, about 14.9, about 20.1, about 21.5, about 22.5, about 22.7, about 28.0, and about 30.1 degrees 2 θ. In further embodiments, the crystalline composition comprising about 70% by weight of D-metyrosine and about 30% by weight of L-metyrosine is characterized by an XRPD pattern comprising three or more peaks at about 10.0, about 20.1, about 22.5, about 22.7, and about 28.0 degrees 2 θ. In yet other embodiments, the crystalline composition comprising about 70% by weight of D-metyrosine and about 30% by weight of L-metyrosine is characterized by an XRPD pattern comprising five or more peaks at about 10.0, about 20.1, about 22.5, about 22.7, and about 28.0 degrees 2 θ.

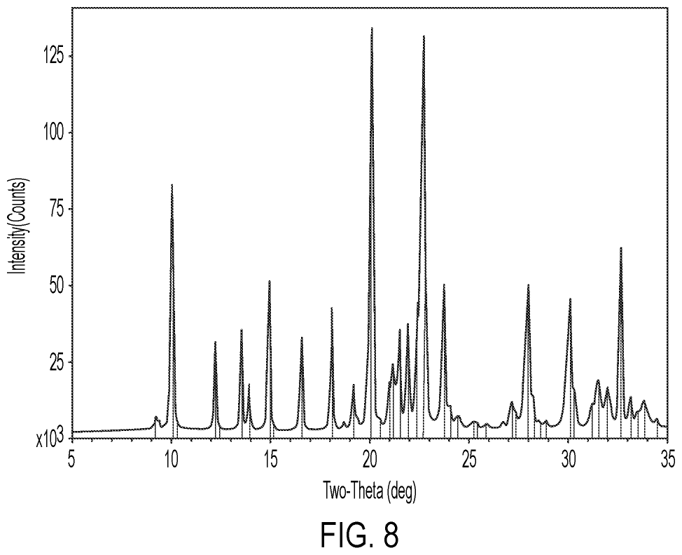

The XRPD of the crystalline composition comprising about 75% by weight of D-metyrosine and about 25% by weight of L-metyrosine shown in FIG. 8 comprises reflection angles (degrees 2 θ), line spacings (d values), and relative intensities as shown in Table 8 or 8A:

TABLE 8

| 2-Theta ° | d(Å) | H % |
|---|---|---|
| 9.2 | 9.6 | 2.8 |
| 10.1 | 8.8 | 61.2 |
| 10.2 | 8.6 | 2.4 |
| 12.3 | 7.2 | 22.1 |
| 12.4 | 7.1 | 1.1 |
| 13.6 | 6.5 | 25.2 |
| 13.9 | 6.3 | 11.2 |
| 15.0 | 5.9 | 37.8 |
| 15.1 | 5.9 | 2 |
| 16.6 | 5.3 | 23.5 |
| 18.1 | 4.9 | 30.4 |
| 19.2 | 4.6 | 10.1 |
| 20.2 | 4.4 | 100 |
| 20.6 | 4.3 | 1.5 |
| 21.1 | 4.2 | 10.3 |
| 21.3 | 4.2 | 15.2 |
| 21.6 | 4.1 | 23.9 |
| 22.0 | 4.0 | 25.9 |
| 22.5 | 3.9 | 30.7 |
| 22.8 | 3.9 | 98.1 |
| 23.8 | 3.7 | 35.1 |
| 24.2 | 3.7 | 4.6 |
| 24.5 | 3.6 | 2 |
| 25.3 | 3.5 | 1.3 |
| 25.5 | 3.5 | 1.3 |
| 26.0 | 3.4 | 0.8 |
| 27.2 | 3.3 | 6.9 |
| 27.4 | 3.3 | 4.5 |
| 28.0 | 3.2 | 36.3 |
| 28.3 | 3.2 | 8.4 |
| 28.6 | 3.1 | 1.8 |
| 29.0 | 3.1 | 1.9 |
| 30.1 | 3.0 | 32 |
| 30.3 | 2.9 | 9.8 |
| 31.3 | 2.9 | 5.7 |
| 31.6 | 2.8 | 11.4 |
| 32.0 | 2.8 | 9.7 |
| 32.7 | 2.7 | 44.8 |
| 33.2 | 2.7 | 7.4 |
| 33.5 | 2.7 | 3.5 |
| 33.8 | 2.6 | 6.7 |
| 34.5 | 2.6 | 2 |

TABLE 8A

| 2-Theta ° | d(Å) | H % |
|---|---|---|
| 10.1 | 8.8 | 61.2 |
| 12.3 | 7.2 | 22.1 |
| 13.6 | 6.5 | 25.2 |
| 13.9 | 6.3 | 11.2 |
| 15.0 | 5.9 | 37.8 |
| 16.6 | 5.3 | 23.5 |
| 18.1 | 4.9 | 30.4 |
| 20.2 | 4.4 | 100 |
| 21.3 | 4.2 | 15.2 |
| 21.6 | 4.1 | 23.9 |
| 22.0 | 4.0 | 25.9 |
| 22.5 | 3.9 | 30.7 |
| 22.8 | 3.9 | 98.1 |
| 23.8 | 3.7 | 35.1 |
| 28.0 | 3.2 | 36.3 |
| 30.1 | 3.0 | 32 |
| 31.6 | 2.8 | 11.4 |
| 32.7 | 2.7 | 44.8 |

The crystalline composition comprising about 75% by weight of D-metyrosine and about 25% by weight of L-metyrosine is characterized by an XRPD pattern comprising a peak at one of the angles listed in Table 8 or 8A. In some embodiments, the crystalline composition comprising about 75% by weight of D-metyrosine and about 25% by weight of L-metyrosine is characterized by an XRPD pattern comprising more than one peak at one of the angles listed in Table 8 or 8A. In other embodiments, the crystalline composition comprising about 75% by weight of D-metyrosine and about 25% by weight of L-metyrosine is characterized by an XRPD pattern comprising two peaks selected from the angles listed in Table 8 or 8A. In further embodiments, the crystalline composition comprising about 75% by weight of D-metyrosine and about 25% by weight of L-metyrosine is characterized by an XRPD pattern comprising three peaks selected from the angles listed in Table 8 or 8A. In yet other embodiments, the crystalline composition comprising about 75% by weight of D-metyrosine and about 25% by weight of L-metyrosine is characterized by an XRPD pattern comprising four peaks selected from the angles listed in Table 8 or 8A. In still further embodiments, the crystalline composition comprising about 75% by weight of D-metyrosine and about 25% by weight of L-metyrosine is characterized by an XRPD pattern comprising five peaks selected from the angles listed in Table 8 or 8A. In other embodiments, the crystalline composition comprising about 75% by weight of D-metyrosine and about 25% by weight of L-metyrosine is characterized by an XRPD pattern comprising six peaks selected from the angles listed in Table 8 or 8A. In further embodiments, the crystalline composition comprising about 75% by weight of D-metyrosine and about 25% by weight of L-metyrosine is characterized by an XRPD pattern comprising seven peaks selected from the angles listed in Table 8 or 8A. In still other embodiments, the crystalline composition comprising about 75% by weight of D-metyrosine and about 25% by weight of L-metyrosine is characterized by an XRPD pattern comprising eight peaks selected from the angles listed in Table 8 or 8A. In yet further embodiments, the crystalline composition comprising about 75% by weight of D-metyrosine and about 25% by weight of L-metyrosine is characterized by an XRPD pattern comprising nine peaks selected from the angles listed in Table 8 or 8A. In other embodiments, the crystalline composition comprising about 75% by weight of D-metyrosine and about 25% by weight of L-metyrosine is characterized by an XRPD pattern comprising ten peaks selected from the angles listed in Table 8 or 8A. In yet further embodiments, the crystalline composition comprising about 75% by weight of D-metyrosine and about 25% by weight of L-metyrosine is characterized by an XRPD pattern comprising more than ten peaks selected from the angles listed in Table 8 or 8A.

In some embodiments, the crystalline composition comprising about 75% by weight of D-metyrosine and about 25% by weight of L-metyrosine is characterized by an XRPD pattern comprising three or more peaks at about 10.1, about 15.0, about 18.1, about 20.2, about 22.5, about 22.8, about 23.8, about 28.0, about 30.1, and about 32.7 degrees 2 θ. In other embodiments, the crystalline composition comprising about 75% by weight of D-metyrosine and about 25% by weight of L-metyrosine is characterized by an XRPD pattern comprising five or more peaks at about 10.1, about 15.0, about 18.1, about 20.2, about 22.5, about 22.8, about 23.8, about 28.0, about 30.1, and about 32.7 degrees 2 θ. In further embodiments, the crystalline composition comprising about 75% by weight of D-metyrosine and about 25% by weight of L-metyrosine is characterized by an XRPD pattern comprising three or more peaks at about 10.1, about 15.0, about 20.2, about 22.8, about 28.0, and about 32.7 degrees 2 θ. In yet other embodiments, the crystalline composition comprising about 75% by weight of D-metyrosine and about 25% by weight of L-metyrosine is characterized by an XRPD pattern comprising five or more peaks at about 10.1, about 15.0, about 20.2, about 22.8, about 28.0, and about 32.7 degrees 2 θ.

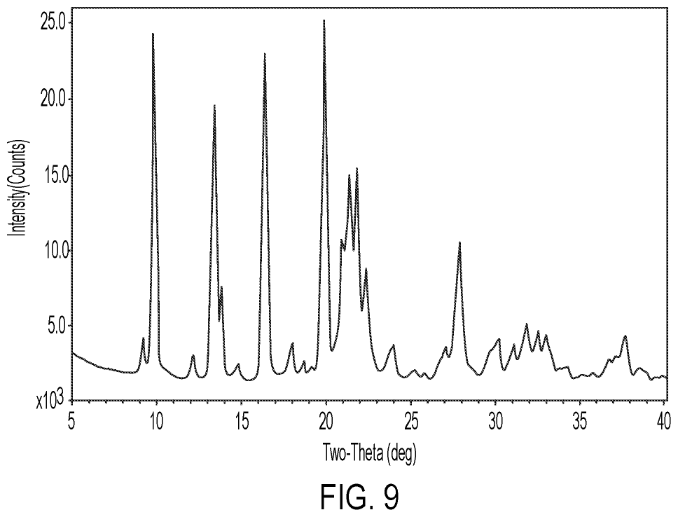

The XRPD of the crystalline composition comprising about 55% by weight of L-metyrosine and about 45% by weight of D-metyrosine shown in FIG. 9 comprises reflection angles (degrees 2 θ±0.2 degrees 2 θ), line spacings (d values), and relative intensities as shown in Table 9 or 9A:

TABLE 9

| 2-Theta ° | Intensity | Intensity % |
|---|---|---|
| 9.2 | 2453 | 10.5 |
| 9.9 | 22545 | 96.2 |
| 12.1 | 1441 | 6.1 |
| 13.4 | 18079 | 77.1 |
| 13.8 | 5885 | 25.1 |
| 14.8 | 979 | 4.2 |
| 16.4 | 21439 | 91.5 |
| 18.0 | 2081 | 8.9 |
| 18.6 | 832 | 3.5 |
| 19.1 | 480 | 2 |
| 20.0 | 23441 | 100 |
| 21.0 | 8828 | 37.7 |
| 21.4 | 13021 | 55.6 |
| 21.9 | 13585 | 58 |
| 22.4 | 7038 | 30 |
| 24.0 | 2097 | 8.9 |
| 25.2 | 524 | 2.2 |
| 25.8 | 277 | 1.2 |
| 27.1 | 1970 | 8.4 |
| 27.9 | 8859 | 37.8 |
| 29.8 | 1721 | 7.3 |
| 30.2 | 2353 | 10 |
| 31.1 | 1949 | 8.3 |
| 31.9 | 3364 | 14.4 |
| 32.6 | 2956 | 12.6 |
| 33.1 | 2724 | 11.6 |
| 34.3 | 721 | 3.1 |
| 35.2 | 163 | 0.7 |
| 35.9 | 404 | 1.7 |
| 36.8 | 1215 | 5.2 |
| 37.2 | 1503 | 6.4 |
| 37.8 | 2733 | 11.7 |
| 38.6 | 736 | 3.1 |
| 40.1 | 262 | 1.1 |

TABLE 9A

| 2-Theta ° | Intensity | Intensity % |
|---|---|---|
| 9.9 | 22545 | 96.2 |
| 13.4 | 18079 | 77.1 |
| 13.8 | 5885 | 25.1 |
| 16.4 | 21439 | 91.5 |
| 20.0 | 23441 | 100 |
| 21.0 | 8828 | 37.7 |
| 21.4 | 13021 | 55.6 |
| 21.9 | 13585 | 58 |
| 22.4 | 7038 | 30 |
| 27.9 | 8859 | 37.8 |
| 31.9 | 3364 | 14.4 |
| 32.6 | 2956 | 12.6 |
| 33.1 | 2724 | 11.6 |
| 37.8 | 2733 | 11.7 |

The crystalline composition comprising about 55% by weight of L-metyrosine and about 45% by weight of D-metyrosine is characterized by an XRPD pattern comprising a peak at one of the angles listed in Table 9 or 9A. In some embodiments, the crystalline composition comprising about 55% by weight of L-metyrosine and about 45% by weight of D-metyrosine is characterized by an XRPD pattern comprising more than one peak at one of the angles listed in Table 9 or 9A. In other embodiments, the crystalline composition comprising about 55% by weight of L-metyrosine and about 45% by weight of D-metyrosine is characterized by an XRPD pattern comprising two peaks selected from the angles listed in Table 9 or 9A. In further embodiments, the crystalline composition comprising about 55% by weight of L-metyrosine and about 45% by weight of D-metyrosine is characterized by an XRPD pattern comprising three peaks selected from the angles listed in Table 9 or 9A. In yet other embodiments, the crystalline composition comprising about 55% by weight of L-metyrosine and about 45% by weight of D-metyrosine is characterized by an XRPD pattern comprising four peaks selected from the angles listed in Table 9 or 9A. In still further embodiments, the crystalline composition comprising about 55% by weight of L-metyrosine and about 45% by weight of D-metyrosine is characterized by an XRPD pattern comprising five peaks selected from the angles listed in Table 9 or 9A. In other embodiments, the crystalline composition comprising about 55% by weight of L-metyrosine and about 45% by weight of D-metyrosine is characterized by an XRPD pattern comprising six peaks selected from the angles listed in Table 9 or 9A. In further embodiments, the crystalline composition comprising about 55% by weight of L-metyrosine and about 45% by weight of D-metyrosine is characterized by an XRPD pattern comprising seven peaks selected from the angles listed in Table 9 or 9A. In still other embodiments, the crystalline composition comprising about 55% by weight of L-metyrosine and about 45% by weight of D-metyrosine is characterized by an XRPD pattern comprising eight peaks selected from the angles listed in Table 9 or 9A. In yet further embodiments, the crystalline composition comprising about 55% by weight of L-metyrosine and about 45% by weight of D-metyrosine is characterized by an XRPD pattern comprising nine peaks selected from the angles listed in Table 9 or 9A. In other embodiments, the crystalline composition comprising about 55% by weight of L-metyrosine and about 45% by weight of D-metyrosine is characterized by an XRPD pattern comprising ten peaks selected from the angles listed in Table 9 or 9A. In yet further embodiments, the crystalline composition comprising about 55% by weight of L-metyrosine and about 45% by weight of D-metyrosine is characterized by an XRPD pattern comprising more than ten peaks selected from the angles listed in Table 9 or 9A.

In some embodiments, the crystalline composition comprising about 55% by weight of L-metyrosine and about 45% by weight of D-metyrosine is characterized by an XRPD pattern comprising three or more peaks at about 9.9, about 13.4, about 16.4, about 20.0, about 21.0, about 21.4, about 21.9, and about 27.9 degrees 2 θ. In other embodiments, the crystalline composition comprising about 55% by weight of L-metyrosine and about 45% by weight of D-metyrosine is characterized by an XRPD pattern comprising five or more peaks at about 9.2, about 9.9, about 13.4, about 16.4, about 20.0, about 21.0, about 21.4, about 21.9, and about 27.9 degrees 2 θ. In further embodiments, the crystalline composition comprising about 55% by weight of L-metyrosine and about 45% by weight of D-metyrosine is characterized by an XRPD pattern comprising three or more peaks at about 9.9, about 13.4, about 16.4, about 20.0, about 21.4, and about 21.9 degrees 2 θ. In yet other embodiments, the crystalline composition comprising about 55% by weight of L-metyrosine and about 45% by weight of D-metyrosine is characterized by an XRPD pattern comprising five or more peaks at about 9.9, about 13.4, about 16.4, about 20.0, about 21.4, about and about 21.9 degrees 2 θ.

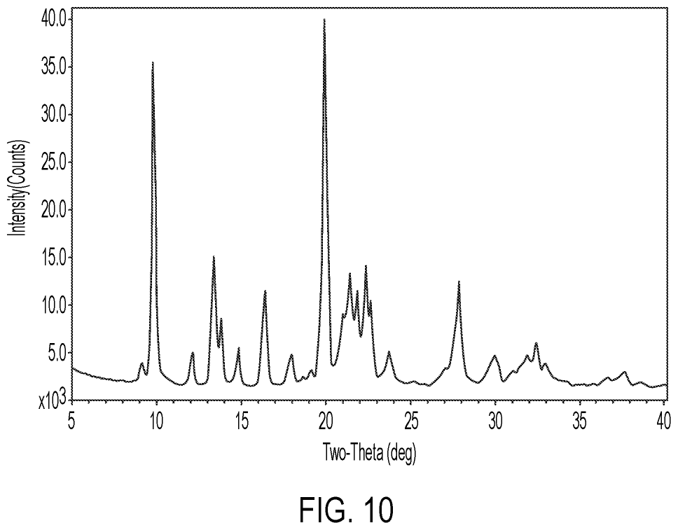

The XRPD of the crystalline composition comprising about 60% by weight of L-metyrosine and about 40% by weight of D-metyrosine shown in FIG. 10 comprises reflection angles (degrees 2 θ±0.2 degrees 2 θ), line spacings (d values), and relative intensities as shown in Table 10 or 10A:

TABLE 10

| 2-Theta ° | Intensity | Intensity % |
|---|---|---|
| 9.2 | 2030 | 5.3 |
| 9.9 | 33600 | 88.5 |
| 12.1 | 3318 | 8.7 |
| 13.4 | 13535 | 35.7 |
| 13.8 | 6889 | 18.1 |
| 14.8 | 3955 | 10.4 |
| 16.4 | 9840 | 25.9 |
| 17.9 | 2978 | 7.8 |
| 18.7 | 590 | 1.6 |
| 19.1 | 1283 | 3.4 |
| 20.0 | 37963 | 100 |
| 21.1 | 6882 | 18.1 |
| 21.5 | 11255 | 29.6 |
| 21.9 | 9455 | 24.9 |
| 22.4 | 11980 | 31.6 |
| 22.7 | 8592 | 22.6 |
| 23.7 | 3257 | 8.6 |
| 25.2 | 345 | 0.9 |
| 25.9 | 165 | 0.4 |
| 27.1 | 1769 | 4.7 |
| 27.9 | 10750 | 28.3 |
| 30.0 | 2972 | 7.8 |
| 31.1 | 1219 | 3.2 |
| 31.5 | 1821 | 4.8 |
| 31.9 | 2936 | 7.7 |
| 32.5 | 4179 | 11 |
| 33. | 2085 | 5.5 |
| 36.7 | 863 | 2.3 |
| 37.7 | 1506 | 4 |
| 38.6 | 424 | 1.1 |
| 40.1 | 215 | 0.6 |

TABLE 10A

| 2-Theta ° | Intensity | Intensity % |
|---|---|---|
| 9.9 | 33600 | 88.5 |
| 13.4 | 13535 | 35.7 |
| 13.8 | 6889 | 18.1 |
| 16.4 | 9840 | 25.9 |
| 20.0 | 37963 | 100 |
| 21.1 | 6882 | 18.1 |
| 21.5 | 11255 | 29.6 |
| 21.9 | 9455 | 24.9 |
| 22.4 | 11980 | 31.6 |
| 22.7 | 8592 | 22.6 |
| 27.9 | 10750 | 28.3 |
| 32.5 | 4179 | 11 |

The crystalline composition comprising about 60% by weight of L-metyrosine and about 40% by weight of D-metyrosine is characterized by an XRPD pattern comprising a peak at one of the angles listed in Table 10 or 10A. In some embodiments, the crystalline composition comprising about 60% by weight of L-metyrosine and about 40% by weight of D-metyrosine is characterized by an XRPD pattern comprising more than one peak at one of the angles listed in Table 10 or 10A. In other embodiments, the crystalline composition comprising about 60% by weight of L-metyrosine and about 40% by weight of D-metyrosine is characterized by an XRPD pattern comprising two peaks selected from the angles listed in Table 10 or 10A. In further embodiments, the crystalline composition comprising about 60% by weight of L-metyrosine and about 40% by weight of D-metyrosine is characterized by an XRPD pattern comprising three peaks selected from the angles listed in Table 10 or 10A. In yet other embodiments, the crystalline composition comprising about 60% by weight of L-metyrosine and about 40% by weight of D-metyrosine is characterized by an XRPD pattern comprising four peaks selected from the angles listed in Table 10 or 10A. In still further embodiments, the crystalline composition comprising about 60% by weight of L-metyrosine and about 40% by weight of D-metyrosine characterized by an XRPD pattern comprising five peaks selected from the angles listed in Table 10 or 10A. In other embodiments, the crystalline composition comprising about 60% by weight of L-metyrosine and about 40% by weight of D-metyrosine is characterized by an XRPD pattern comprising six peaks selected from the angles listed in Table 10 or 10A. In further embodiments, the crystalline composition comprising about 60% by weight of L-metyrosine and about 40% by weight of D-metyrosine is characterized by an XRPD pattern comprising seven peaks selected from the angles listed in Table 10 or 10A. In still other embodiments, the crystalline composition comprising about 60% by weight of L-metyrosine and about 40% by weight of D-metyrosine is characterized by an XRPD pattern comprising eight peaks selected from the angles listed in Table 10 or 10A. In yet further embodiments, the crystalline composition comprising about 60% by weight of L-metyrosine and about 40% by weight of D-metyrosine is characterized by an XRPD pattern comprising nine peaks selected from the angles listed in Table 10 or 10A. In other embodiments, the crystalline composition comprising about 60% by weight of L-metyrosine and about 40% by weight of D-metyrosine is characterized by an XRPD pattern comprising ten peaks selected from the angles listed in Table 10 or 10A. In yet further embodiments, the crystalline composition comprising about 60% by weight of L-metyrosine and about 40% by weight of D-metyrosine is characterized by an XRPD pattern comprising more than ten peaks selected from the angles listed in Table 10 or 10A.

In some embodiments, the crystalline composition comprising about 60% by weight of L-metyrosine and about 40% by weight of D-metyrosine is characterized by an XRPD pattern comprising three or more peaks at about 9.9, about 13.4, about 16.4, about 20.0, about 21.5, about 21.9, about 22.4, about 22.7, and about 27.9 degrees 2 θ. In other embodiments, the crystalline composition comprising about 60% by weight of L-metyrosine and about 40% by weight of D-metyrosine is characterized by an XRPD pattern comprising five or more peaks at about 9.9, about 13.4, about 16.4, about 20.0, about 21.5, about 21.9, about 22.4, about 22.7, and about 27.9 degrees 2 θ. In further embodiments, the crystalline composition comprising about 60% by weight of L-metyrosine and about 40% by weight of D-metyrosine is characterized by an XRPD pattern comprising three or more peaks at about 9.9, about 13.4, about 20.0, about 21.5, about 22.4, and about 27.9 degrees 2 θ. In yet other embodiments, the crystalline composition comprising about 60% by weight of L-metyrosine and about 40% by weight of D-metyrosine is characterized by an XRPD pattern comprising five or more peaks at about 9.9, about 13.4, about 20.0, about 21.5, about 22.4, and about 27.9 degrees 2 θ.

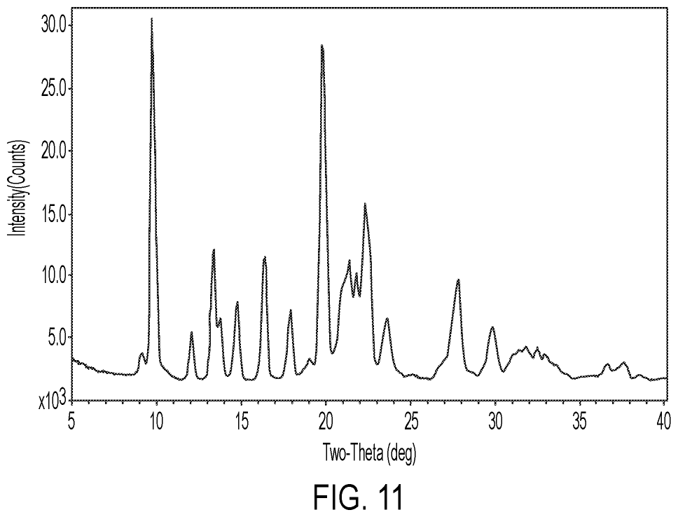

The XRPD of the crystalline composition comprising about 65% by weight of L-metyrosine and about 35% by weight of D-metyrosine shown in FIG. 11 comprises reflection angles (degrees 2 θ±0.2 degrees 2 θ), line spacings (d values), and relative intensities as shown in Table 11 or 11A:

TABLE 11

| 2-Theta ° | Intensity | Intensity % |
|---|---|---|
| 9.2 | 1767 | 6.1 |
| 9.9 | 28868 | 100 |
| 12.1 | 3791 | 13.1 |
| 13.4 | 10363 | 35.9 |
| 13.8 | 4908 | 17 |
| 14.8 | 6208 | 21.5 |
| 16.4 | 9808 | 34 |
| 17.9 | 5391 | 18.7 |
| 19.1 | 1435 | 5 |
| 20.0 | 26494 | 91.8 |
| 21.0 | 7290 | 25.3 |
| 21.4 | 9041 | 31.3 |
| 21.8 | 8031 | 27.8 |
| 22.4 | 13731 | 47.6 |
| 22.6 | 10730 | 37.2 |
| 23.7 | 4567 | 15.8 |
| 25.1 | 350 | 1.2 |
| 27.1 | 1534 | 5.3 |
| 27.9 | 7777 | 26.9 |
| 29.9 | 3835 | 13.3 |
| 31.1 | 1597 | 5.5 |
| 31.9 | 2421 | 8.4 |
| 32.5 | 2221 | 7.7 |
| 33.0 | 1779 | 6.2 |
| 36.7 | 1135 | 3.9 |
| 37.7 | 1304 | 4.5 |
| 38.6 | 417 | 1.4 |

TABLE 11A

| 2-Theta ° | Intensity | Intensity % |
|---|---|---|
| 9.9 | 28868 | 100 |
| 12.1 | 3791 | 13.1 |
| 13.4 | 10363 | 35.9 |
| 13.8 | 4908 | 17 |
| 14.8 | 6208 | 21.5 |
| 16.4 | 9808 | 34 |
| 17.9 | 5391 | 18.7 |
| 20.0 | 26494 | 91.8 |
| 21.0 | 7290 | 25.3 |
| 21.4 | 9041 | 31.3 |
| 21.8 | 8031 | 27.8 |
| 22.4 | 13731 | 47.6 |
| 22.6 | 10730 | 37.2 |
| 23.7 | 4567 | 15.8 |
| 27.9 | 7777 | 26.9 |
| 29.9 | 3835 | 13.3 |

The crystalline composition comprising about 65% by weight of L-metyrosine and about 35% by weight of D-metyrosine is characterized by an XRPD pattern comprising a peak at one of the angles listed in Table 11 or 11A. In some embodiments, the crystalline composition comprising about 65% by weight of L-metyrosine and about 35% by weight of D-metyrosine is characterized by an XRPD pattern comprising more than one peak at one of the angles listed in Table 11 or 11A. In other embodiments, the crystalline composition comprising about 65% by weight of L-metyrosine and about 35% by weight of D-metyrosine is characterized by an XRPD pattern comprising two peaks selected from the angles listed in Table 11 or 11A. In further embodiments, the crystalline composition comprising about 65% by weight of L-metyrosine and about 35% by weight of D-metyrosine is characterized by an XRPD pattern comprising three peaks selected from the angles listed in Table 11 or 11A. In yet other embodiments, the crystalline composition comprising about 65% by weight of L-metyrosine and about 35% by weight of D-metyrosine is characterized by an XRPD pattern comprising four peaks selected from the angles listed in Table 11 or 11A. In still further embodiments, the crystalline composition comprising about 65% by weight of L-metyrosine and about 35% by weight of D-metyrosine is characterized by an XRPD pattern comprising five peaks selected from the angles listed in Table 11 or 11A. In other embodiments, the crystalline composition comprising about 65% by weight of L-metyrosine and about 35% by weight of D-metyrosine is characterized by an XRPD pattern comprising six peaks selected from the angles listed in Table 11 or 11A. In further embodiments, the crystalline composition comprising about 65% by weight of L-metyrosine and about 35% by weight of D-metyrosine is characterized by an XRPD pattern comprising seven peaks selected from the angles listed in Table 11 or 11A. In still other embodiments, the crystalline composition comprising about 65% by weight of L-metyrosine and about 35% by weight of D-metyrosine is characterized by an XRPD pattern comprising eight peaks selected from the angles listed in Table 11 or 11A. In yet further embodiments, the crystalline composition comprising about 65% by weight of L-metyrosine and about 35% by weight of D-metyrosine is characterized by an XRPD pattern comprising nine peaks selected from the angles listed in Table 11 or 11A. In other embodiments, the crystalline composition comprising about 65% by weight of L-metyrosine and about 35% by weight of D-metyrosine is characterized by an XRPD pattern comprising ten peaks selected from the angles listed in Table 11 or 11A. In yet further embodiments, the crystalline composition comprising about 65% by weight of L-metyrosine and about 35% by weight of D-metyrosine is characterized by an XRPD pattern comprising more than ten peaks selected from the angles listed in Table 11 or 11A.

In some embodiments, the crystalline composition comprising about 65% by weight of L-metyrosine and about 35% by weight of D-metyrosine is characterized by an XRPD pattern comprising three or more peaks at about 9.9, about 13.4, about 14.8, about 16.4, about 20.0, about 21.0, about 21.4, about 21.8, about 22.4, about 22.6, and about 27.9 degrees 2 θ. In other embodiments, the crystalline composition comprising about 65% by weight of L-metyrosine and about 35% by weight of D-metyrosine is characterized by an XRPD pattern comprising five or more peaks at about 9.9, about 13.4, about 14.8, about 16.4, about 20.0, about 21.0, about 21.4, about 21.8, about 22.4, about 22.6, and about 27.9 degrees 2 θ. In further embodiments, the crystalline composition comprising about 65% by weight of L-metyrosine and about 35% by weight of D-metyrosine is characterized by an XRPD pattern comprising three or more peaks at about 9.9, about 13.4, about 16.4, about 20.0, about 21.4, about 22.4, and about 22.6 degrees 2 θ. In yet other embodiments, the crystalline composition comprising about 65% by weight of L-metyrosine and about 35% by weight of D-metyrosine is characterized by an XRPD pattern comprising five or more peaks at about 9.9, about 13.4, about 16.4, about 20.0, about 21.4, about 22.4, and about 22.6 degrees 2 θ.

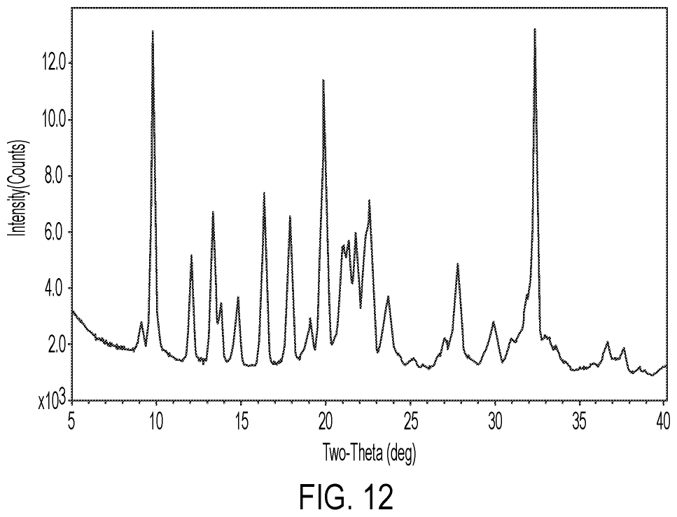

The XRPD of the crystalline composition comprising about 70% by weight of L-metyrosine and about 30% by weight of D-metyrosine shown in FIG. 12 comprises reflection angles (degrees 2 θ±0.2 degrees 2 θ), line spacings (d values), and relative intensities as shown in Table 12 or 12A:

TABLE 12

| 2-Theta ° | Intensity | Intensity % |
|---|---|---|
| 9.2 | 1060 | 8.9 |
| 9.9 | 11501 | 96.1 |
| 12.1 | 3747 | 31.3 |
| 13.4 | 5339 | 44.6 |
| 13.8 | 2085 | 17.4 |
| 14.8 | 2413 | 20.2 |
| 16.4 | 6116 | 51.1 |
| 18.0 | 5181 | 43.3 |
| 19.1 | 1475 | 12.3 |
| 20.0 | 9952 | 83.2 |
| 21.1 | 4103 | 34.3 |
| 21.4 | 4243 | 35.5 |
| 21.9 | 4497 | 37.6 |
| 22.4 | 4495 | 37.6 |
| 22.7 | 5648 | 47.2 |
| 23.7 | 2335 | 19.5 |
| 24.4 | 384 | 3.2 |
| 25.2 | 302 | 2.5 |
| 25.8 | 136 | 1.1 |
| 27.1 | 1071 | 9 |
| 27.9 | 3647 | 30.5 |
| 30.0 | 1554 | 13 |
| 31.1 | 962 | 8 |
| 32.5 | 11964 | 100 |
| 33.0 | 1174 | 9.8 |
| 33.3 | 1063 | 8.9 |
| 33.7 | 853 | 7.1 |
| 34.3 | 271 | 2.3 |
| 35.1 | 65.5 | 0.5 |
| 36.0 | 275 | 2.3 |
| 36.7 | 1069 | 8.9 |
| 37.7 | 877 | 7.3 |
| 38.6 | 259 | 2.2 |
| 40.2 | 209 | 1.7 |

TABLE 12A

| 2-Theta ° | Intensity | Intensity % |
|---|---|---|
| 9.9 | 11501 | 96.1 |
| 12.1 | 3747 | 31.3 |
| 13.4 | 5339 | 44.6 |
| 13.8 | 2085 | 17.4 |
| 14.8 | 2413 | 20.2 |
| 16.4 | 6116 | 51.1 |
| 18.0 | 5181 | 43.3 |
| 19.1 | 1475 | 12.3 |
| 20.0 | 9952 | 83.2 |
| 21.1 | 4103 | 34.3 |
| 21.4 | 4243 | 35.5 |
| 21.9 | 4497 | 37.6 |
| 22.4 | 4495 | 37.6 |
| 22.7 | 5648 | 47.2 |
| 23.7 | 2335 | 19.5 |
| 27.9 | 3647 | 30.5 |
| 30.0 | 1554 | 13 |
| 32.5 | 11964 | 100 |

The crystalline composition comprising about 70% by weight of L-metyrosine and about 30% by weight of D-metyrosine composition comprising is characterized by an XRPD pattern comprising a peak at one of the angles listed in Table 12 or 12A. In some embodiments, the crystalline composition comprising about 70% by weight of L-metyrosine and about 30% by weight of D-metyrosine is characterized by an XRPD pattern comprising more than one peak at one of the angles listed in Table 12 or 12A. In other embodiments, the crystalline composition comprising about 70% by weight of L-metyrosine and about 30% by weight of D-metyrosine is characterized by an XRPD pattern comprising two peaks selected from the angles listed in Table 12 or 12A. In further embodiments, the crystalline composition comprising about 70% by weight of L-metyrosine and about 30% by weight of D-metyrosine is characterized by an XRPD pattern comprising three peaks selected from the angles listed in Table 12 or 12A. In yet other embodiments, the crystalline composition comprising about 70% by weight of L-metyrosine and about 30% by weight of D-metyrosine is characterized by an XRPD pattern comprising four peaks selected from the angles listed in Table 12 or 12A. In still further embodiments, the crystalline composition comprising about 70% by weight of L-metyrosine and about 30% by weight of D-metyrosine is characterized by an XRPD pattern comprising five peaks selected from the angles listed in Table 12 or 12A. In other embodiments, the crystalline composition comprising about 70% by weight of L-metyrosine and about 30% by weight of D-metyrosine is characterized by an XRPD pattern comprising six peaks selected from the angles listed in Table 12 or 12A. In further embodiments, the crystalline composition comprising about 70% by weight of L-metyrosine and about 30% by weight of D-metyrosine is characterized by an XRPD pattern comprising seven peaks selected from the angles listed in Table 12 or 12A. In still other embodiments, the crystalline composition comprising about 70% by weight of L-metyrosine and about 30% by weight of D-metyrosine is characterized by an XRPD pattern comprising eight peaks selected from the angles listed in Table 12 or 12A. In yet further embodiments, the crystalline composition comprising about 70% by weight of L-metyrosine and about 30% by weight of D-metyrosine is characterized by an XRPD pattern comprising nine peaks selected from the angles listed in Table 12 or 12A. In other embodiments, the crystalline composition comprising about 70% by weight of L-metyrosine and about 30% by weight of D-metyrosine is characterized by an XRPD pattern comprising ten peaks selected from the angles listed in Table 12 or 12A. In yet further embodiments, the crystalline composition comprising about 70% by weight of L-metyrosine and about 30% by weight of D-metyrosine characterized by an XRPD pattern comprising more than ten peaks selected from the angles listed in Table 12 or 12A.

In some embodiments, the crystalline composition comprising about 70% by weight of L-metyrosine and about 30% by weight of D-metyrosine is characterized by an XRPD pattern comprising three or more peaks at about 9.9, about 12.1, about 13.4, about 16.4, about 18.0, about 20.0, about 21.1, about 21.4, about 21.9, about 22.4, about 22.7, about 27.9, and about 32.5 degrees 2 θ. In other embodiments, the crystalline composition comprising about 70% by weight of L-metyrosine and about 30% by weight of D-metyrosine is characterized by an XRPD pattern comprising five or more peaks at about 9.9, about 12.1, about 13.4, about 16.4, about 18.0, about 20.0, about 21.1, about 21.4, about 21.9, about 22.4, about 22.7, about 27.9, and about 32.5 degrees 2 θ. In further embodiments, the crystalline composition comprising about 70% by weight of L-metyrosine and about 30% by weight of D-metyrosine is characterized by an XRPD pattern comprising three or more peaks at about 9.9, about 13.4, about 16.4, about 18.0, about 20.0, about 22.7, and about 32.5 degrees 2 θ. In yet other embodiments, the crystalline composition comprising about 70% by weight of L-metyrosine and about 30% by weight of D-metyrosine is characterized by an XRPD pattern comprising five or more peaks at about 9.9 about, about 13.4, about 16.4, about 18.0, about 20.0, about 22.7, and about 32.5 degrees 2 θ.

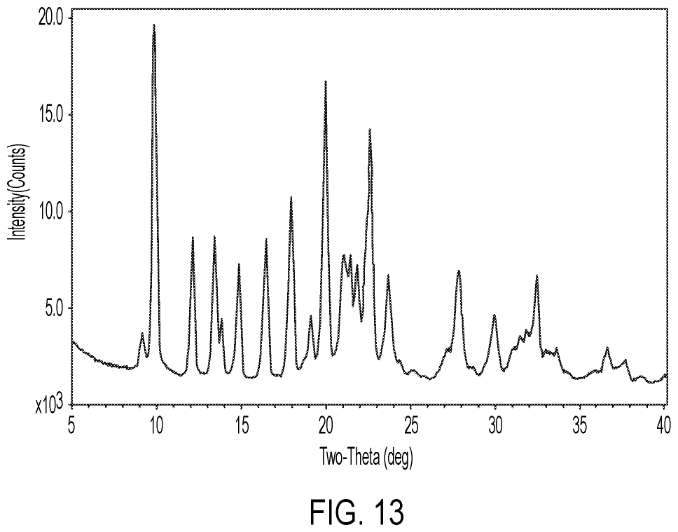

The XRPD of the crystalline composition comprising about 75% by weight of L-metyrosine and about 25% by weight of D-metyrosine shown in FIG. 13 comprises reflection angles (degrees 2 θ±degrees 2 θ), line spacings (d values), and relative intensities as shown in Table 13 or 13A:

TABLE 13

| 2-Theta ° | Intensity | Intensity % |
|---|---|---|
| 9.2 | 1907 | 10.6 |
| 9.9 | 17908 | 100 |
| 12.1 | 7135 | 39.8 |
| 13.4 | 7202 | 40.2 |
| 13.8 | 2952 | 16.5 |
| 14.8 | 5852 | 32.7 |
| 16.5 | 7055 | 39.4 |
| 18.0 | 9138 | 51 |
| 19.1 | 3012 | 16.8 |
| 20.0 | 15048 | 84 |
| 21.1 | 6129 | 34.2 |
| 21.5 | 6069 | 33.9 |
| 21.9 | 5555 | 31 |
| 22.4 | 7065 | 39.4 |
| 22.7 | 12595 | 70.3 |
| 23.7 | 5136 | 28.7 |
| 24.4 | 856 | 4.8 |
| 25.2 | 379 | 2.1 |
| 25.9 | 154 | 0.9 |
| 27.9 | 5430 | 30.3 |
| 30.0 | 3083 | 17.2 |
| 31.5 | 2041 | 11.4 |
| 31.9 | 2326 | 13 |
| 32.6 | 5179 | 28.9 |
| 33.1 | 1390 | 7.8 |
| 33.7 | 1525 | 8.5 |
| 36.7 | 1684 | 9.4 |
| 37.8 | 1005 | 5.6 |
| 38.7 | 332 | 1.9 |
| 40.2 | 273 | 1.5 |

TABLE 13A

| 2-Theta ° | Intensity | Intensity % |
|---|---|---|
| 9.9 | 17908 | 100 |
| 12.1 | 7135 | 39.8 |
| 13.4 | 7202 | 40.2 |
| 13.8 | 2952 | 16.5 |
| 14.8 | 5852 | 32.7 |
| 16.5 | 7055 | 39.4 |
| 18.0 | 9138 | 51 |
| 19.1 | 3012 | 16.8 |
| 20.0 | 15048 | 84 |
| 21.1 | 6129 | 34.2 |
| 21.5 | 6069 | 33.9 |
| 21.9 | 5555 | 31 |
| 22.4 | 7065 | 39.4 |
| 22.7 | 12595 | 70.3 |
| 23.7 | 5136 | 28.7 |
| 27.9 | 5430 | 30.3 |
| 30.0 | 3083 | 17.2 |
| 31.5 | 2041 | 11.4 |
| 31.9 | 2326 | 13 |
| 32.6 | 5179 | 28.9 |

The crystalline composition comprising about 75% by weight of L-metyrosine and about 25% by weight of D-metyrosine is characterized by an XRPD pattern comprising a peak at one of the angles listed in Table 13 or 13A. In some embodiments, the crystalline composition comprising about 75% by weight of L-metyrosine and about 25% by weight of D-metyrosine is characterized by an XRPD pattern comprising more than one peak at one of the angles listed in Table 13 or 13A. In other embodiments, the crystalline composition comprising about 75% by weight of L-metyrosine and about 25% by weight of D-metyrosine is characterized by an XRPD pattern comprising two peaks selected from the angles listed in Table 13 or 13A. In further embodiments, the crystalline composition comprising about 75% by weight of L-metyrosine and about 25% by weight of D-metyrosine is characterized by an XRPD pattern comprising three peaks selected from the angles listed in Table 13 or 13A. In yet other embodiments, the crystalline composition comprising about 75% by weight of L-metyrosine and about 25% by weight of D-metyrosine is characterized by an XRPD pattern comprising four peaks selected from the angles listed in Table 13 or 13A. In still further embodiments, the crystalline composition comprising about 75% by weight of L-metyrosine and about 25% by weight of D-metyrosine is characterized by an XRPD pattern comprising five peaks selected from the angles listed in Table 13 or 13A. In other embodiments, the crystalline composition comprising about 75% by weight of L-metyrosine and about 25% by weight of D-metyrosine is characterized by an XRPD pattern comprising six peaks selected from the angles listed in Table 13 or 13A. In further embodiments, the crystalline composition comprising about 75% by weight of L-metyrosine and about 25% by weight of D-metyrosine is characterized by an XRPD pattern comprising seven peaks selected from the angles listed in Table 13 or 13A. In still other embodiments, the crystalline composition comprising about 75% by weight of L-metyrosine and about 25% by weight of D-metyrosine is characterized by an XRPD pattern comprising eight peaks selected from the angles listed in Table 13 or 13A. In yet further embodiments, the crystalline composition comprising about 75% by weight of L-metyrosine and about 25% by weight of D-metyrosine is characterized by an XRPD pattern comprising nine peaks selected from the angles listed in Table 13 or 13A. In other embodiments, the crystalline composition comprising about 75% by weight of L-metyrosine and about 25% by weight of D-metyrosine is characterized by an XRPD pattern comprising ten peaks selected from the angles listed in Table 13 or 13A. In yet further embodiments, the crystalline composition comprising about 75% by weight of L-metyrosine and about 25% by weight of D-metyrosine is characterized by an XRPD pattern comprising more than ten peaks selected from the angles listed in Table 13 or 13A.

In some embodiments, the crystalline composition comprising about 75% by weight of L-metyrosine and about 25% by weight of D-metyrosine is characterized by an XRPD pattern comprising three or more peaks at about 9.9, about 12.1, about 13.4, about 14.8, about 16.5, about 18.0, about 20.0, about 21.1, about 21.5, about 21.9, about 22.4, about 22.7, and about 27.9 degrees 2 θ. In other embodiments, the crystalline composition comprising about 75% by weight of L-metyrosine and about 25% by weight of D-metyrosine is characterized by an XRPD pattern comprising five or more peaks at about 9.9, about 12.1, about 13.4, about 14.8, about 16.5, about 18.0, about 20.0, about 21.1, about 21.5, about 21.9, about 22.4, about 22.7, and about 27.9 degrees 2 θ. In further embodiments, the crystalline composition comprising about 75% by weight of L-metyrosine and about 25% by weight of D-metyrosine is characterized by an XRPD pattern comprising three or more peaks at about 9.9, about 13.4, about 18.0, about 20.0, and about 22.7 degrees 2 θ. In yet other embodiments, the crystalline composition comprising about 75% by weight of L-metyrosine and about 25% by weight of D-metyrosine is characterized by an XRPD pattern comprising five or more peaks at about 9.9, about 13.4, about 18.0, about 20.0, and about 22.7 degrees 2 θ.

### Preparation Methods

Also provided by the disclosure are methods preparing the crystalline compositions described herein. In some embodiments, the methods include agitating a solution comprising a racemic mixture of metyrosine enantiomers in methyl tert-butyl ether for at least about 60 minutes under conditions effective to precipitate a first solid. In some embodiments, the solution is agitated for at least about 90 minutes, about 2 hours, about 3 hours, about 6 hours, about 9 hours, or at least about 12 hours. Following precipitation, at least a portion of the first solid is isolated using techniques known in the art. Once isolated, an amount of the first solid is mixed with an amount of D-metyrosine or L-metyrosine in methyl tert-butyl ether for at least about 60 minutes under conditions effective to precipitate a second solid. In some embodiments, the solution is mixed for at least about 90 minutes, about 2 hours, about 3 hours, about 6 hours, about 9 hours, or at least about 12 hours. The second solid is then isolated using techniques known in the art. The weight ratio of the first solid to the D-metyrosine or L-metyrosine is at least about 5:about. In some embodiments, the weight ratio of the first solid and the D-metyrosine or L-metyrosine is about 1:about 1. In other embodiments, the weight ratio of the first solid and the D-metyrosine or L-metyrosine is about 2:about 1. In further embodiments, the weight ratio of the first solid and the D-metyrosine or L-metyrosine is about 3:about 1. In yet other embodiments, the weight ratio of the first solid and the D-metyrosine or L-metyrosine is about 4:about 1. In still further embodiments, the weight ratio of the first solid and the D-metyrosine or L-metyrosine is about 6:about 1. In other embodiments, the weight ratio of the first solid and the D-metyrosine or L-metyrosine is about 7:about 1. In further embodiments, the weight ratio of the first solid and the D-metyrosine or L-metyrosine is about 8:about 1. In still other embodiments, the weight ratio of the first solid and the D-metyrosine or L-metyrosine is about 9:about 1.

In other embodiments, the methods include agitating a solution comprising D-metyrosine and L-metyrosine in methyl tert-butyl ether for at least about 60 minutes under conditions effective to precipitate a first solid. In some embodiments, the solution is agitated for at least about 90 minutes, about 2 hours, about 3 hours, about 6 hours, about 9 hours, or at least about 12 hours. Following precipitation, at least a portion of the first solid is isolated using techniques known in the art. Once isolated, an amount of the first solid is mixed with an amount of D-metyrosine or L-metyrosine in methyl tert-butyl ether for at least about 60 minutes under conditions effective to precipitate a second solid. In some embodiments, the solution is mixed for at least about 90 minutes, about 2 hours, about 3 hours, about 6 hours, about 9 hours, or at least about 12 hours. The second solid is then isolated using techniques known in the art.

In further embodiments, the methods include agitating a solution containing D-metyrosine or L-metyrosine, racemic metyrosine, an aqueous acid and water (3V), following by heating to about 45 to about 55° C. In some aspects, the solution is heated to about 50° C. In other aspects, the aqueous acid is aqueous HCl. The heating is continued until dissolution of the solid material, which may be determined by one skilled in the art. In other aspects, the heating is continued for 30 minutes or more. After heating, the heated solution is cooled to about 15 to about 25° C. In some embodiments, the solution is cooled to about 20° C. Following cooling, the pH is adjusted to about 5 to about 6 using a base. In some embodiments, the base is ammonium hydroxide, such as 28-30% ammonium hydroxide. The pH is desirably adjusted at elevated temperatures such as a temperature of about 435 to about 55° C. In some aspects, the solution is heated to about 50° C. The slurry is then cooled to about 5 to about 15° C. and retained at this temperature. In some embodiments, the slurry is cooled to about 10° C. In other aspects, the slurry is retained at the cooled temperature for about 1 hour. The solid was isolated and washed. In some embodiment, the solid was isolated using filtration. In other embodiments, the solid was washed with water.

Isolation techniques utilized as described in the disclosure include, without limitation, filtering, decanting, centrifuging, among others.

Additional steps may be performed in order to provide the crystalline composition. In some embodiments, the methods further comprise drying the first solid, the second solid, or a combination thereof. One of skill in the art would readily understand suitable techniques for drying the solids. Such drying techniques include, without limitation, vacuum drying optionally in the presence of heat, oven drying, air drying, among others.

### Pharmaceutical Formulations

Pharmaceutical formulations useful herein, in some embodiments, contain one or more crystalline compositions discussed herein in a pharmaceutically acceptable carrier or diluent with other optional suitable pharmaceutically inert or inactive ingredients.

The pharmaceutical formulations include one or more crystalline compositions discussed herein formulated neat or with one or more pharmaceutical carriers for administration, the proportion of which is determined by the solubility and chemical nature of crystalline composition, chosen route of administration and standard pharmacological practice. The pharmaceutical carrier may be solid or liquid.

Although the one or more crystalline compositions discussed herein may be administered alone, it may also be administered in the presence of one or more pharmaceutical carriers that are physiologically compatible. The carriers may be in dry or liquid form and must be pharmaceutically acceptable. Liquid pharmaceutical formulations are typically sterile solutions or suspensions.

When liquid carriers are utilized, they are desirably sterile liquids. Liquid carriers are typically utilized in preparing solutions, suspensions, emulsions, syrups and elixirs. In one embodiment, the one or more crystalline compositions discussed herein is dissolved a liquid carrier. In another embodiment, one or more crystalline compositions discussed herein is suspended in a liquid carrier. One of skill in the art of formulations would be able to select a suitable liquid carrier, depending on the route of administration. In one embodiment, the liquid carrier includes, without limitation, water, organic solvents, oils, fats, or mixtures thereof. In another embodiment, the liquid carrier is water containing cellulose derivatives such as sodium carboxymethyl cellulose. In a further embodiment, the liquid carrier is water and/or dimethylsulfoxide. Examples of organic solvents include, without limitation, alcohols such as monohydric alcohols and polyhydric alcohols, e.g., glycols and their derivatives, among others. Examples of oils include, without limitation, fractionated coconut oil, arachis oil, corn oil, peanut oil, and sesame oil and oily esters such as ethyl oleate and isopropyl myristate.

Alternatively, one or more crystalline compositions discussed herein may be formulated in a solid carrier. In one embodiment, the pharmaceutical formulations may be compacted into a unit dose form, i.e., tablet or caplet. In another embodiment, the pharmaceutical formulations may be added to unit dose form, i.e., a capsule. In a further embodiment, the pharmaceutical formulations may be formulated for administration as a powder. The solid carrier may perform a variety of functions, i.e., may perform the functions of two or more of the excipients described below. For example, the solid carrier may also act as a flavoring agent, lubricant, solubilizer, suspending agent, filler, glidant, compression aid, binder, disintegrant, or encapsulating material. Suitable solid carriers include, without limitation, calcium phosphate, dicalcium phosphate, magnesium stearate, talc, starch, sugars (including, e.g., lactose and sucrose), cellulose (including, e.g., microcrystalline cellulose, methyl cellulose, sodium carboxymethyl cellulose), polyvinylpyrrolidine, low melting waxes, ion exchange resins, and kaolin. The solid carrier can contain other suitable excipients, including those described below.

Examples of excipients which may be combined with one or more crystalline compositions discussed herein include, without limitation, adjuvants, antioxidants, binders, buffers, coatings, coloring agents, compression aids, diluents, disintegrants, emulsifiers, emollients, encapsulating materials, fillers, flavoring agents, glidants, granulating agents, lubricants, metal chelators, osmo-regulators, pH adjustors, preservatives, solubilizers, sorbents, stabilizers, sweeteners, surfactants, suspending agents, syrups, thickening agents, or viscosity regulators. See, the excipients described in the “Handbook of Pharmaceutical Excipients”, 5th Edition, Eds.: Rowe, Sheskey, and Owen, APhA Publications (Washington, DC), Dec. 14, 2005, which is incorporated herein by reference.

### Therapeutic Methods/Uses

The crystalline compositions described herein are useful in treating a number of maladies which may be treated using metyrosine by administering the crystalline composition to a subject in need thereof.

As used herein, the terms “treat” and “treatment” refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As employed above and throughout the disclosure the term “effective amount” refers to an amount effective, at dosages, and for periods of time necessary, to achieve the desired result with respect to the treatment of the relevant disorder, condition, or side effect. It will be appreciated that the effective amount of the crystalline composition described herein will vary from patient to patient not only with crystalline composition, the route of administration, and the ability of the components to elicit a desired result in the individual. Other factors which may affect the effective amount includes, without limitation, the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage being at the discretion of the attending physician. Dosage regimes may be adjusted to provide the improved therapeutic response. An effective amount is also one in which any toxic or detrimental effects of the components are outweighed by the therapeutically beneficial effects. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. In some embodiments, the subject is a human.

“Administration” to a subject is not limited to any particular delivery system and may include, without limitation, oral (for example, in capsules, suspensions or tablets), parenteral (including subcutaneous, intravenous, intramedullary, intraarticular, intramuscular, or intraperitoneal injection), topical, or transdermal. Administration to a host may occur in a single dose or in repeat administrations, and in any of a variety of physiologically acceptable salt forms, and/or with an acceptable pharmaceutical carrier and/or additive as part of a pharmaceutical composition (described earlier). Once again, physiologically acceptable salt forms and standard pharmaceutical formulation techniques are well known to persons skilled in the art (see, for example, Remington's Pharmaceutical Sciences, Mack Publishing Co.).

In some embodiments, the crystalline compositions may be used to treat cancer in a patient in need thereof. Examples of cancers that may be treated include, without limitation, breast cancer, pancreatic cancer, prostate cancer, thyroid cancer, lung cancer, colon cancer, brain cancer, glioma, ovarian cancer, bile duct cancer, cholangiocarcinoma, sarcoma, Hodgkin's lymphoma, lymphoma, renal cancer, tonsil squamous cell carcinoma, or colorectal cancer.

In further embodiments, the crystalline compositions may be used to treat conditions linked with “leaky gut syndrome”.

In other embodiments, the crystalline compositions may be used to treat intestinal hyperpermeability.

In further embodiments, the crystalline compositions described herein are useful in treating hyperglycemia.

In yet other embodiments, the crystalline compounds herein may be used to treat diabetes, autism, fibromyalgi a, inflammatory bowel disease (IBD), graft versus host disease (GVHD), HIV/ AIDS, multiple organ dysfunction syndrome, irritable bowel syndrome (IBS), celiac disease, eczema, psoriasis, acute pancreatitis, Parkinson's disease, depression, chronic fatigue syndrome, asthma, multiple sclerosis, arthritis, ankylosing spondylitis, nonalcoholic fatty liver disease, alcoholic cirrhosis, environmental enteropathy, or kwashiorkor, or combinations thereof,

The crystalline composition described here may be administered only once, or it may be administered multiple times. For multiple dosages, the composition may be, for example, administered three times a day, twice a day, once a day, once every two days, twice a week, weekly, once every two weeks, or monthly.

The dosage of the crystalline composition described herein may range from about 1 mg to about 4 g, In some embodiments, the dosage may range from about 10 mg to about 1500 mg. In other embodiments, the dosage is about 1. about 5, about 25, about 50, about 100, about 125, about 150, about 175, about 200, about 250, about 300, about 350, about 400, about 450, about 500, about 750, about 1000, about 1500, about 1750, about 2000, about 2250, about 2500, about 2750, about 3000, about 3250, about 3500, about 3750, or about 4000 mg.

In the following examples, efforts have been made to ensure accuracy with respect to numbers used (e.g. amounts, temperature, etc) but some experimental error and deviation should be accounted for Unless indicated otherwise, temperature is in degrees C., pressure is at or near atmospheric.

### Aspects

Aspect 1. A crystalline composition comprising about 55% to about 95% by weight of D-metyrosine and about 5% to about 45% by weight of L-metyrosine, based on the total weight of the crystalline composition, and is characterized by a powder X-ray diffraction pattern comprising at least three peaks selected from about 10.2 degrees 2 θ, about 13.5 degrees 2 θ, about 20.1 degrees 2 θ, about 21.5 degrees 2 θ, about 22.5 degrees 2 θ, or about 28.0 degrees 2 θ.

Aspect 2. The crystalline composition of Aspect 1, comprising at least four of the peaks.

Aspect 3. The crystalline composition of Aspect 1, comprising at least five of the peaks.

Aspect 4. The crystalline composition of any one of the preceding Aspects, further comprising one or more peaks at about 16.6 degrees 2 θ, about 22.0 degrees 2 θ, or about 32.6 degrees 2 θ.

Aspect 5. The crystalline composition of any one of Aspects 1 to 4, comprising about 55 to about 75% by weight of D-metyrosine and about 25% to about 45% by weight of L-metyrosine.

Aspect 6. The crystalline composition of any one of the preceding Aspects, comprising about 55% by weight of D-metyrosine and about 45% by weight of L-metyrosine.

Aspect 7. The crystalline composition of any one of Aspects 1 to 4, comprising about 60% by weight of D-metyrosine and about 40% by weight of L-metyrosine.

Aspect 8. The crystalline composition of any one of Aspects 1 to 4, comprising about 65% by weight of D-metyrosine and about 35% by weight of L-metyrosine.

Aspect 9. The crystalline composition of any one of Aspects 1 to 4, comprising about 70% by weight of D-metyrosine and about 30% by weight of L-metyrosine.

Aspect 10. The crystalline composition of any one of Aspects 1 to 4, comprising about 80% by weight of D-metyrosine and about 20% by weight of L-metyrosine.

Aspect 11. The crystalline composition of any one of Aspects 1 to 4, comprising about 85% by weight of D-metyrosine and about 15% by weight of L-metyrosine.

Aspect 12. The crystalline composition of any one of Aspects 1 to 4, comprising about 90% by weight of D-metyrosine and about 10% by weight of L-metyrosine.

Aspect 13. The crystalline composition of any one of Aspects 1 to 4, comprising about 95% by weight of D-metyrosine and about 5% by weight of L-metyrosine.

Aspect 14. A crystalline composition comprising about 55% to about 95% by weight of L-metyrosine and about 5% to about 45% by weight of D-metyrosine, based on the total weight of the crystalline composition, and characterized a powder X-ray diffraction pattern comprising at least three peaks selected from about 9.9 degrees 2 θ, about 13.4 degrees 2 θ, about 16.4 degrees 2 θ, about 20.0 degrees 2 θ, or about 21.9 degrees 2 θ.

Aspect 15. The crystalline composition of Aspect 17, comprising at least four of the peaks.

Aspect 16. The crystalline composition of Aspect 17, comprising at least five of the peaks.

Aspect 17. The crystalline composition of any one of Aspects 17 to 19, further comprising one or more peaks at about 13.8 degrees 2 θ, about 21.0 degrees 2 θ, about 21.4 degrees 2 θ, about 22.4 degrees 2 θ, about 27.9 degrees 2 θ, or about 32.6 degrees 2 θ.

Aspect 18. The crystalline composition of any one of Aspects 17 to 20, comprising about 55 to about 75% by weight of L-metyrosine and about 25% to about 45% by weight of D-metyrosine.

Aspect 19. The crystalline composition of any one of Aspects 17 to 20, comprising about 55% by weight of L-metyrosine and about 45% by weight of D-metyrosine.

Aspect 20. The crystalline composition of any one of Aspects 17 to 20, comprising about 60% by weight of L-metyrosine and about 40% by weight of D-metyrosine.

Aspect 21. The crystalline composition of any one of Aspects 17 to 20, comprising about 65% by weight of L-metyrosine and about 35% by weight of D-metyrosine.

Aspect 22. The crystalline composition of any one of Aspects 17 to 20, comprising about 70% by weight of L-metyrosine and about 30% by weight of D-metyrosine.

Aspect 23. The crystalline composition of any one of Aspects 17 to 20, comprising about 80% by weight of L-metyrosine and about 20% by weight of D-metyrosine.

Aspect 24. The crystalline composition of any one of Aspects 17 to 20, comprising about 85% by weight of L-metyrosine and about 15% by weight of D-metyrosine.

Aspect 25. The crystalline composition of any one of Aspects 17 to 20, comprising about 90% by weight of L-metyrosine and about 10% by weight of D-metyrosine.

Aspect 26. The crystalline composition of any one of Aspects 17 to 20, comprising about 95% by weight of L-metyrosine and about 5% by weight of D-metyrosine.

Aspect 27. A pharmaceutical formulation comprising a crystalline composition of any one of the preceding Aspects and a pharmaceutically acceptable excipient.

Aspect 28. A method for treating cancer in a patient in need thereof, the method comprising administering to the patient a composition of any one of Aspects 1 to 34 or a pharmaceutical formulation of Aspect 35.

Aspect 29. The method of Aspect 36, wherein the cancer is breast cancer, pancreatic cancer, prostate cancer, thyroid cancer, lung cancer, colon cancer, brain cancer, glioma, ovarian cancer, bile duct cancer, cholangiocarcinoma, sarcoma, Hodgkin's lymphoma, lymphoma, renal cancer, tonsil squamous cell carcinoma, or colorectal cancer.

Aspect 30. A method for preparing a composition of Aspect 1, comprising:
agitating a solution comprising a racemic mixture of metyrosine enantiomers in methyl tert-butyl ether for at least about 60 minutes under conditions effective to precipitate a first solid;
isolating at least a portion of the first solid;
mixing an amount of the first solid with an amount of D-metyrosine or L-metyrosine in methyl tert-butyl ether for at least about 60 minutes under conditions effective to precipitate a second solid; and
isolating at least a portion of the second solid.

Aspect 31. The method of Aspect 38, further comprising drying the first solid, the second solid, or a combination thereof

Aspect 32. The method of Aspect 38 or 39, wherein the weight ratio of the first solid and the D-metyrosine or L-metyrosine is about 9:about 1.

Aspect 33. The method of Aspect 38 or 39, wherein the weight ratio of the first solid and the D-metyrosine or L-metyrosine is about 8:about 1.

Aspect 34. The method of Aspect 38 or 39, wherein the weight ratio of the first solid and the D-metyrosine or L-metyrosine is about 7:about 1.

Aspect 35. The method of Aspect 38 or 39, wherein the weight ratio of the first solid and the D-metyrosine or L-metyrosine is about 6:about 1.

Aspect 36. The method of Aspect 38 or 39, wherein the weight ratio of the first solid and the D-metyrosine or L-metyrosine is about 5:about 1.

### EXAMPLES

### Example 1

### Preparation of Racemic α-Methyltyrosine

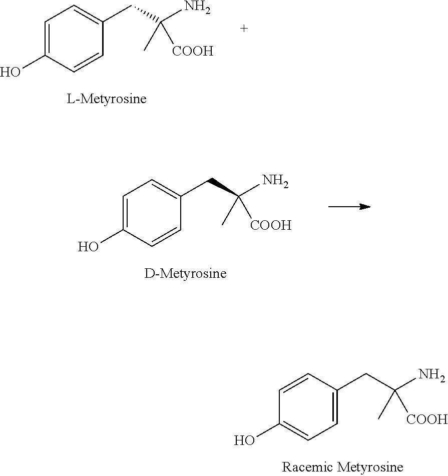

To a 3-L glass reactor was charged 125 g of L-metyrosine and D-metyrosine each and MTBE (9V). The slurry was agitated for ≥60 minutes at room temperature, upon which the solids were isolated via filtration. The cake was washed with MTBE (2×2V) and the solids were dried under vacuum at ≤45° C. to provide racemic metyrosine α-metyrosine (249.73 g; 99.9% yield). Chiral HPLC: D 50.1% and L 49.9%.

### Example 2

### Preparation of Crystalline of D/L-Enriched α-Methyltyrosine

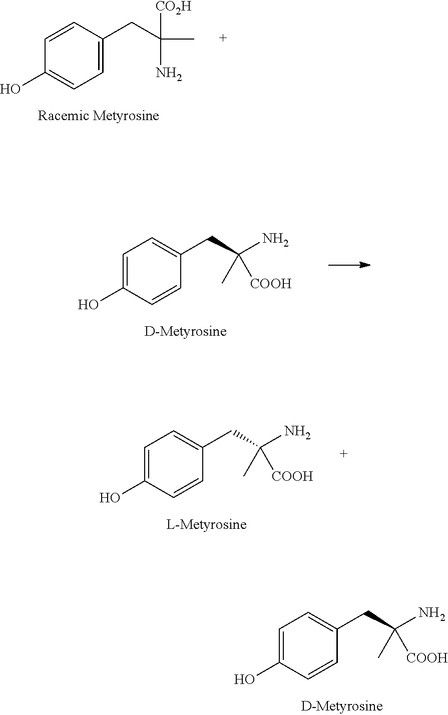

A 100-mL 3N round bottom flask was charged with 2.5 g of the racemic metyrosine of Example 1 and 2.5 g of L-metyrosine or D-metyrosine to provide 5 g of a metyrosine blend. MTBE (5V) was then added to the flask and a slurry was produced upon agitation. The slurry was agitated for ≥60 minutes at room temperature, upon which the solids were isolated via filtration. The cake was washed with MTBE (2×2V) and the solids were dried under vacuum at <45° C.

### Example 3

### Preparation of Crystalline of D/L-enriched α-Methyltyrosine

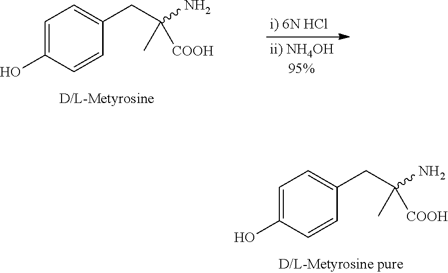

A glass reactor was charged with D- and/or L-enriched metyrosine (5 g) from Example 2, 6N aq. HCl (4.0V) and water (3V). The slurry was then heated to 50±5° C. Upon dissolution, the reaction mixture was allowed to age for >30 minutes. The heat was turned off and the reaction was allowed to cool to 20±5° C. The pH was then adjusted to 5-6 at 50±5° C. using 28-30% ammonium hydroxide. The slurry was then cooled to 10±5° C. and aged for 1 hour. The solids were isolated via filtration and the cake washed with water (4V). See, Table 14 for the starting ratios and final ratios.

TABLE 14

| | Amount | |
|---|---|---|
| Sample | Recovered (g) | Final Composition |
| 1 | 4.60 | 50.2% D/49.8% L |
| 2 | 4.69 | 54.5% D/45.5% L |
| 3 | 5.04 | 60.1% D/39.9% L |
| 4 | 4.94 | 64.6% D/35.4% L |
| 5 | 4.66 | 69.1% D/30.9% L |
| 6 | 4.59 | 75.2% D/24.8% L |
| 7 | 4.56 | 54.0% L/46.0% D |
| 8 | 5.01 | 59.5% L/40.5% D |
| 9 | 4.87 | 64.3% L/35.7% D |
| 10 | 5.72 | 69.2% L/30.8% D |
| 11 | 5.43 | 74.8% L/25.2% D |

XRPD patterns for all samples were then obtained. For the D-metyrosine enriched samples (samples 1-6), XRPD analyzed was performed using a Rigaku Ultima IV diffractometer with a fixed sample stage, together with the parameters of Table 15.

TABLE 15

| Source | Cu Kα1 1.54060 A | |
|---|---|---|
| Voltage | 40 | kV |
| Current | 44 | mA |
| Scan range 2θ | 5-35 | |
| Step size | 0.02 | degrees |
| Time/step | 1.2 | s/step |

| | |
|---|---|
| Goniometer type | TH/TH |
| Detector | DTex silicon strip detector with |
| | a divergence of ⅔ degree |

Lens optics

| | | |
|---|---|---|
| Divergence slit | 10 | mm |
| Scattering slit | 13 | mm |
| Receiving slit | 13 | mm |

For the samples of enriched L-metyrosine, XRPD was obtained using a D8 Advance diffractometer and radiation reflection goniometer stage, together with the parameters of Table 16.

TABLE 16

| Source | Cu Kα1 1.54060 A | |
|---|---|---|
| Voltage | 40 | kV |
| Current | 40 | mA |
| Scan range 2θ | 5-40 | |
| Step size | 0.0144 | |
| Time/step | 1 | s |
| Goniometer type | TH/TH | |
| Sample rotation speed | 30 | rpm |

| | |
|---|---|
| Detector | PSD Vantec-1, Use default |
| | electronic window 12 |

Lens optics

| Divergence slit | 0.6 | mm |
|---|---|---|
| Detector slit | 10.09 | mm |
| Primary Soller slit | 2.5 | |
| Secondary Soller slit | 2.5 | |
| Antiscattering slit | 6.65 | mm |

XRPD patterns are shown in FIGS. 1-13.

The contents of all references, patent applications, patents, and published patent applications, as well as the Figures, cited throughout this application are hereby incorporated by reference.

Those skilled in the art will recognize, or be able to ascertain using no more than routine experimentation, many equivalents to the specific embodiments of the disclosure described herein. Such equivalents are intended to be encompassed by the following claims.

What is claimed is:

1. A crystalline composition comprising about 55% to about 95% by weight of D-metyrosine and about 5% to about 45% by weight of L-metyrosine, based on the total weight of the crystalline composition, and is characterized by a powder X-ray diffraction pattern comprising at least three peaks selected from about 10.2 degrees 2 θ, about 13.5 degrees 2 θ, about 20.1 degrees 2 θ, about 21.5 degrees 2 θ, about 22.5 degrees 2 θ, or about 28.0 degrees 2 θ.

2. The crystalline composition of claim 1, comprising at least four of the peaks.

3. The crystalline composition of claim 1, comprising at least five of the peaks.

4. The crystalline composition of claim 1, further comprising one or more peaks at about

16. 6 degrees 2 θ, about 22.0 degrees 2 θ, or about 32.6 degrees 2 θ.

5. The crystalline composition of claim 1, comprising about 55 to about 75% by weight of D-metyrosine and about 25% to about 45% by weight of L-metyrosine.

6. The crystalline composition of claim 1, comprising about 55% by weight of D-metyrosine and about 45% by weight of L-metyrosine.

7. The crystalline composition of claim 1, comprising about 60% by weight of D-metyrosine and about 40% by weight of L-metyrosine.

8. The crystalline composition of claim 1, comprising about 65% by weight of D-metyrosine and about 35% by weight of L-metyrosine.

9. The crystalline composition of claim 1, comprising about 70% by weight of D-metyrosine and about 30% by weight of L-metyrosine.

10. The crystalline composition of claim 1, comprising about 80% by weight of D-metyrosine and about 20% by weight of L-metyrosine.

11. The crystalline composition of claim 1, comprising about 85% by weight of D-metyrosine and about 15% by weight of L-metyrosine.

12. The crystalline composition of claim 1, comprising about 90% by weight of D-metyrosine and about 10% by weight of L-metyrosine.

13. The crystalline composition of claim 1, comprising about 95% by weight of D-metyrosine and about 5% by weight of L-metyrosine.

14. A crystalline composition comprising about 55% to about 95% by weight of L-metyrosine and about 5% to about 45% by weight of D-metyrosine, based on the total weight of the crystalline composition, and characterized a powder X-ray diffraction pattern comprising at least three peaks selected from about 9.9 degrees 2 θ, about 13.4 degrees 2 θ, about 16.4 degrees 2 θ, about 20.0 degrees 2 θ, or about 21.9 degrees 2 θ.

15. The crystalline composition of claim 14, comprising at least four of the peaks.

16. The crystalline composition of claim 14, comprising at least five of the peaks.

17. The crystalline composition of claim 14, further comprising one or more peaks at about 13.8 degrees 2 θ, about 21.0 degrees 2 θ, about 21.4 degrees 2 θ, about 22.4 degrees 2 θ, about 27.9 degrees 2 θ, or about 32.6 degrees 2 θ.

18. The crystalline composition of claim 14, comprising about 55 to about 75% by weight of L-metyrosine and about 25% to about 45% by weight of D-metyrosine.

19. The crystalline composition of claim 14, comprising about 55% by weight of L-metyrosine and about 45% by weight of D-metyrosine.

20. The crystalline composition of claim 14, comprising about 60% by weight of L-metyrosine and about 40% by weight of D-metyrosine.

21. The crystalline composition of claim 14, comprising about 65% by weight of L-metyrosine and about 35% by weight of D-metyrosine.

22. The crystalline composition of claim 14, comprising about 70% by weight of L-metyrosine and about 30% by weight of D-metyrosine.

23. The crystalline composition of claim 14, comprising about 80% by weight of L-metyrosine and about 20% by weight of D-metyrosine.

24. The crystalline composition of claim 14, comprising about 85% by weight of L-metyrosine and about 15% by weight of D-metyrosine.

25. The crystalline composition of claim 14, comprising about 90% by weight of L-metyrosine and about 10% by weight of D-metyrosine.

26. The crystalline composition of claim 14, comprising about 95% by weight of L-metyrosine and about 5% by weight of D-metyrosine.

27. A pharmaceutical formulation comprising a crystalline composition of claim 1 and a pharmaceutically acceptable excipient.

28. A method for treating cancer in a patient in need thereof, the method comprising administering to the patient a composition of claim 1.

29. The method of claim 28, wherein the cancer is breast cancer, pancreatic cancer, prostate cancer, thyroid cancer, lung cancer, colon cancer, brain cancer, glioma, ovarian cancer, bile duct cancer, cholangiocarcinoma, sarcoma, Hodgkin's lymphoma, lymphoma, renal cancer, tonsil squamous cell carcinoma, or colorectal cancer.

30. A method for preparing a composition of claim 1, comprising:
agitating a solution comprising a racemic mixture of metyrosine enantiomers in methyl tert-butyl ether for at least about 60 minutes under conditions effective to precipitate a first solid;
isolating at least a portion of the first solid;
mixing an amount of the first solid with an amount of D-metyrosine or L-metyrosine in methyl tert-butyl ether for at least about 60 minutes under conditions effective to precipitate a second solid; and
isolating at least a portion of the second solid.

31. The method of claim 30, further comprising drying the first solid, the second solid, or a combination thereof

32. The method of claim 30, wherein the weight ratio of the first solid and the D-metyrosine or L-metyrosine is about 9:about 1.

33. The method of claim 30, wherein the weight ratio of the first solid and the D-metyrosine or L-metyrosine is about 8:about 1.

34. The method of claim 30, wherein the weight ratio of the first solid and the D-metyrosine or L-metyrosine is about 7:about 1.

35. The method of claim 30, wherein the weight ratio of the first solid and the D-metyrosine or L-metyrosine is about 6:about 1.

36. The method of claim 30, wherein the weight ratio of the first solid and the D-metyrosine or L-metyrosine is about 5:about 1.
